SEQUENCE LISTING (9 sequences)

SEQ ID NO: 1 - protein, 31 aa, Lineus longissimus
GCIATGSFCTLSKGCCTKNCGWNFKCNPPNQ

SEQ ID NO: 2 - protein, 31 aa, artificial
  Consensus sequence
  misc_feature (4)..(5): Xaa can be any naturally occurring amino acid
  misc_feature (8)..(8): Xaa can be any naturally occurring amino acid
  misc_feature (11)..(11): Xaa can be any naturally occurring amino acid
  misc_feature (13)..(13): Xaa can be any naturally occurring amino acid
  misc_feature (25)..(25): Xaa can be any naturally occurring amino acid
  misc_feature (28)..(28): Xaa can be any naturally occurring amino acid
GCIXXGSXCTXSXGCCTKNCGWNFXCNXPNQ

SEQ ID NO: 3 - protein, 31 aa, Lineus longissimus
GCIATGSVCTLSKGCCTKNCGWNFKCNPPNQ

SEQ ID NO: 4 - protein, 31 aa, Lineus lacteus
GCIKTGSGCTLSKGCCTKNCGWNFKCNPPNQ

SEQ ID NO: 5 - protein, 31 aa, Lineus sanguieneus
GCISTGSFCTLSKGCCTKNCGWNFKCNPPNQ

SEQ ID NO: 6 - protein, 31 aa, Lineus pseudolacteus
GCIATGSFCTLSKGCCTKNCGWNFHCNPPNQ

SEQ ID NO: 7 - protein, 31 aa, Lineus sanguieneus
GCIKTGSFCTLSKGCCTKNCGWNFHCNPPNQ

SEQ ID NO: 8 - protein, 31 aa, Lineus ruber
GCIPVGSMCTISNGCCTKNCGWNFHCNKPNQ

SEQ ID NO: 9 - protein, 27 aa, Riseriellus occultus
  MISC_FEATURE (1)..(27): Partial sequence truncated at C-terminus.
GCIATGSFCTLSKGCCTKNCGWNFACN

(19) United States
(12) Patent Application Publication
(10) Pub. No.: US 2019/0256564 A1
(43) Pub. Date: Aug. 22, 2019

(54) NEMERTEA-DERIVED BIOACTIVE COMPOUNDS

(71) Applicants: Ulf Göransson, Uppsala (SE); Erik Jacobsson, Uppsala (SE); Håkan Andersson, Kalmar (SE); Malin Strand, 452 35 Strömstad (SE); The University of Queensland, Queensland (AU)

(72) Inventors: Ulf Göransson, Uppsala (SE); Erik Jacobsson, Uppsala (SE); Håkan Andersson, Kalmar (SE); Malin Strand, Strömstad (SE); Johan Rosengren, Wilston (AU)

(21) Appl. No.: 16/313,843

(22) PCT Filed: Jun. 27, 2017
(86) PCT No.: PCT/SE2017/050705

§ 371 Date: Dec. 27, 2018

(30) Foreign Application Priority Data
Jun. 27, 2016 (SE) 1650919-2

(51) Int. Cl. C07K 14/435; A01N 63/02

(52) U.S. Cl. CPC C07K 14/43536; A61K 38/00; A01N 63/02

(57) ABSTRACT

An isolated peptide or peptidomimetic, comprising an α-nemertide moiety, wherein a) said α-nemertide moiety has a sequence according to SEQ ID NO: 1; or b) said α-nemertide moiety has a sequence differing from SEQ ID NO:1 by residue substitutions, deletions or insertions numbering no more than 9 in total. Medical, pest control and research uses thereof.

### TECHNICAL FIELD

The present invention relates to the field of bioactive peptides/peptidomimetics, in biotechnology, medicine and pest control.

### BACKGROUND TO THE INVENTION

Peptides and proteins originating from animal venoms and toxins are intriguing sources of bioactive compounds. Some of these toxins have found their way to the market as drugs or pharmacological tools, and others are finding applications in biotechnology and agriculture. Snakes, scorpions, spiders, lizards, and centipedes are known producers of peptide toxins, but there are other classes of organisms for which the chemistry, biology and ecology largely remains unknown. The inventors have explored one such neglected source of toxins: the nemerteans or ribbon worms.

This, mainly marine, phylum of animals shares similarities with one of the most well studied group of venomous animals, namely the cone snails of family Conidae. Both use a proboscis for capture of prey and/or for defence, and one class of nemerteans is also equipped with a stiletto having the same apparent function as the radula tooth of the cone snail: venom injection. However, it is the mucus that covers the epidermis of nemerteans that appears to be the most conspicuous source of chemistry of these animals. Already in 1900, Wilson reported that a minute drop of the mucus of placed on his tongue resulted in a numbing sensation for several hours (Wilson Q J, Microsc. Sci. 1900, 43, 97-U33). This activity has later been explained by the isolation of low molecular weight toxins from some nemerteans, including anabasine, pyridyl alcohols (Kem W R, Integr. Comp. Biol. 1985, 25, 99-111) and tetrodotoxin (Asakawa M et al, Toxicon 2003, 41, 747-53).

In comparison, the interest for any protein or peptide based toxins from nemerteans has been limited. Reports are confined to the cytolytic 10 kDa “A-toxins”, the 6 kDa neurotoxic “B-toxins” isolated from the mucus of the milky ribbon worm, Cerebratulus lacteus (Kem W R, J. Biol. Chem. 1976, 251, 4184-92), and the 10 kDa parborlysins that were discovered recently in Parborlasia corrugatus (Butala M et al, Toxicon, 2015, 108, 32-7). The molecular targets of these possible peptide toxins are yet unknown.

The phylum of Nemertea comprises approximately 1300 species. One of the more spectacular species is Lineus longissimus, which is known as the longest animal on earth with a body length of up to 30-50 m. It is found in the northern hemisphere, where it lives at the sea bottom from depths of 10 m and below; in some areas it can also be found in the intertidal shores. L. longissimus sparked the inventors' interest as a possible source of tetrodotoxin (Carroll S et al, J. Exp. Mar. Biol. Ecol. 2003, 288, 51-63), but instead lead to the unexpected discovery of the novel compounds disclosed herein.

Thus, an object of the present invention is the provision of improved or alternative compounds affecting voltage-gated sodium channels, other ion channels, or other targets, for use in medicine, veterinary medicine, biotechnology, agriculture, research and the like applications.

### Definitions

The term peptidomimetic in the context of the present application is defined as a peptide-like polymer chain designed to structurally mimic a peptide, but having in some respects different or improved properties.

The term non-natural residue in the context of the present application refer to an amino acid or amino-acid analogue that does not occur in peptides or proteins produced in naturally-occurring organisms, as part of a peptide or peptidomimetic chain.

The term treatment in the present context refers to treatments resulting in a beneficial effect on a subject afflicted with the condition to be treated, including any degree of alleviation, including minor alleviation, substantial alleviation, major alleviation as well as cure. Preferably, the degree of alleviation is at least a minor alleviation.

The term prevention in the present context refers to preventive measures resulting in any degree of reduction in the likelihood of developing the condition to be prevented, including a minor, substantial or major reduction in likelihood of developing the condition as well as total prevention. Preferably, the degree of likelihood reduction is at least a minor reduction.

The term voltage-gated sodium channel in the context of the present application refers to integral membrane proteins than form ion channels conducting sodium ions through a cell's plasma membrane. By definition, open/closed state of voltage-gated channels is normally mainly governed by the voltage potential across the plasma membrane. In humans, there are 9 known types voltage-gated sodium channels, containing type-defining α-subunits termed Naᵥ1.1 through 1.9, in association with a modulating beta-subunit Naᵥβ1-4. A number of known toxins, such as tetrodotoxin, saxotoxin and several conotoxins exert their effects through binding to voltage-gated sodium channels.

### BRIEF DESCRIPTION OF THE FIGURES

FIG. 1. The nemertean Lineus longissimus (A) A specimen of L. longissimus. (B) Both anopla and enopla species have a proboscis, but only enoplan species are equipped with stylets. At the bottom, a simplified phylogenetic tree of Lophotrochozoa, displaying the relationship to mollusca in which the cone snails are found.

FIG. 2. (A) RP-HPLC-UV trace of a high molecular fraction after size exclusion chromatography of the mucus of Lineus longissimus. Nemertides α-1, α-2 and β-1 are marked. (B) MS spectra of the three peptides α-1 (M+H⁺, 3308.35, monoisotopic mass), α-2 (M+H⁺3260.40, monoisotopic mass) and β-1 (M+H⁺6419.00, average mass). Multiply charged ions are marked by their m/z.

FIG. 3. Sequencing of α-nemertides. (A,B) MSMS sequencing of the N-terminal fragments of nemertides α-1 and α-2. N-terminal tryptic fragment showing the substitution of F into V at position 8. The b- and y-ion series where peaks overlap are highlighted in light gray box. The position 8 is boxed. All Cys residues were alkylated using IAM (Cys+57). (C): Ion-map of enzymatically cleaved peptides, *: hydroxyproline. (D): precursor of α-1 with ER-signal, pro-region and mature toxin marked, as predicted by conoprec. **: pre-sequence cleavage site and ***, mature sequence lysine cleavage site. The C-terminal part of the α-1 and α-2 sequences ( . . . CN(Hyp)(Hyp)NQ-COOH) did not give rise to any peaks that could be sequenced in the enzymatic digestion products, nor in the fragmentation of the full peptides. An ion at 487 m/z that is consistent with the Hyp-Hyp-N-Q C-terminal could be found in both the native α-1, 2 and synthetic α-1, and was confirmed by the co-elution experiment. Glu-C cleavage of α-1 and 2 did not result in any fragments, suggesting that no Glu residues are present in the sequences. (E) Results from a BLAST search of available transcriptomes, disulphide connectivity in the sequences are inferred from the nemertide α-1 NMR structure. Grey boxes below the alignment show absolute similarity. Notes: 1 (SEQ ID NO: 1) present in Lineus longissimus, L. lacteus and L. ruber; 2 (SEQ ID NO: 3) L. longissimus, L. ruber; 3 (SEQ ID NO: 4) L. lacteus; L. lacteus, L. pseudolacteus; 4 (SEQ ID NO: 5) L. sanguieneus; 5 (SEQ ID NO: 6) L. pseudolacteus; 6 L. sanguieneus; 7 (SEQ ID NO: 8) L. ruber. One partial sequence was found in R. occultus, 8 (SEQ ID NO: 9).

FIG. 4. Folding, co-injection and dose estimation in Carcinus maenas. (A): HPLC-UV of the folding process from 0 to 16 hours after folding initiation, all traces were recorded at 215 nm. B: UPLC-QToF co-injection of synthetic α-1 (S) and native (N) α-1, the individual traces for single injection of S and N are shown. (C) Table of effective dose estimation of α-1 in C. maenas assay, all injections were made in duplicate. * the injected crabs did not survive in the time frame of the assay. (D) Left: healthy control, injected with sterile filtered seawater, right: typical response to injection with α-1.

FIG. 5. (A) Activity profile of α-1 on vertebrate Naᵥ channels (Naᵥ1.1, Naᵥ1.4, Naᵥ1.5, Naᵥ1.6 and Naᵥ1.8). For each subtype, left panels show representative whole-cell current traces in control and toxin conditions. The dotted line indicates the zero-current level. The asterisk (*) marks steady-state current traces after application of 6 μM toxin. Traces shown are representative traces of a least 3 independent experiments (n≥3). Right panels show steady-state activation (squares) and inactivation (circles) curves in control (open symbols) and toxin conditions (6 μM α-1, closed symbols). (B) Concentration-response curve for Naᵥ1.6 indicating the concentration dependence of the α-1 induced effect.

FIG. 6. (A) Activity profile of α-1 on invertebrate Naᵥ channels of Blattella germanica (BgNaᵥ1), Drosophila melanogaster (DmNaᵥ1), Varroa destructor (VdNaᵥ1). Representative whole-cell current traces in control and toxin conditions are shown. The dotted line indicates the zero-current level. The asterisk (*) marks steady-state current traces after application of 1 μM toxin. Traces shown are representative of at least 3 independent experiments (n≥3). B, Concentration-response curves for BgNav1 indicating the concentration dependence of the α-1 induced effect. The EC50 value was found to be 8.6±2.9 nM (C), Normalized voltage-current relationship. (D) Steady-state activation and inactivation curves in control (open symbols) and toxin conditions (10 nM α-1, closed symbols). No significant alteration of activation was noted since V_{1/2} values yielded −29.1±2.2 mV in control and −32.5±3.2 mV after addition of 10 nM α-1. For the inactivation curves, the V_{1/2} shifted from −60.4±0.6 mV to −54.5±1.6 in control and toxin situation, respectively. (E) Recovery from inactivation in control (open symbols) and in the presence of 10 nM α-1 (closed symbols). V_{1/2} values yielded 11.6±0.4 ms and 3.8±0.5 ms in control and after application of 10 nM α-1, respectively. (F) Both panels show current traces evoked by 50 ms depolarizations of 5 mV from −90 mV to −30 mV in control (left) and after the addition of 10 nM α-1 (right). (H) To investigate the state-dependence of inhibition, the following protocol was used. As control, a series of depolarizing pulses was applied to an oocyte expressing BgNaᵥ channels. Thereafter, 10 nM α-1 was added and no pulsing was performed for 2 minutes. Next, a similar series of pulses was executed. An expected degree of delay of inactivation was observed after the 2 minute incubation, indicating that the open state is not required for toxin interaction with the channel.

FIG. 7. Three dimensional structure of nemertide α-1. (A) Line representation of the 20 models with lowest MolProbity score. (B) Ribbon representation of the model with lowest MolProbity score with disulphides (roman numbers), C and N-terminal labeled. (C) Surface representation of the model in B. Basic residues in blue, nonpolar in green, cystine in yellow and hydroxyprolines in cyan. The aromatic amino acids F8, F22, and W24 are labeled. F8 is the only difference between α-1 and -2. The 20 models in A were superimposed in MOLMOL, and displayed in PyMOL. All other figures were produced in PyMOL. (D) Ribbon representation of nemertide α-1 and its closest structural analogs found in the PDB identified by the Daliserver. Nemertide α-1, liver expressed antimicrobial peptide 2 (LEAP-2, PBD: 2l1q), ω-Atracotoxin-HVIa (ω-ACTX-HV1, PBD: 1axh) and κ-theraphotoxin-Scg1a (SGTx1, PBD: 1la4).

FIG. 8: NMR statistics summarized in Table 1.

FIG. 9. Effect of Nemertides α-1, α-2, α-5 and α-6 in A. salina microwell assay. All values displayed are averages; experiments were performed in duplicate.

FIG. 10. Effect of Nemertides α-1, α-3 and α-4 in A. salina microwell assay. All values displayed are averages; experiments were performed in duplicate.

FIG. 11. Effect of Nemertide α-1 mutants I3A, T5A, S7A, and F8A with control Nemertide α-1 (“Alpha-1”) in A. salina microwell assay. All values displayed are averages; experiments were performed in triplicate.

FIG. 12. Effect of reduced (unfolded) Nemertides α-3 and α-4 with control Nemertide α-1 (“Alpha-1”) in the A. salina assay. All values displayed are averages; experiments were performed in duplicate.

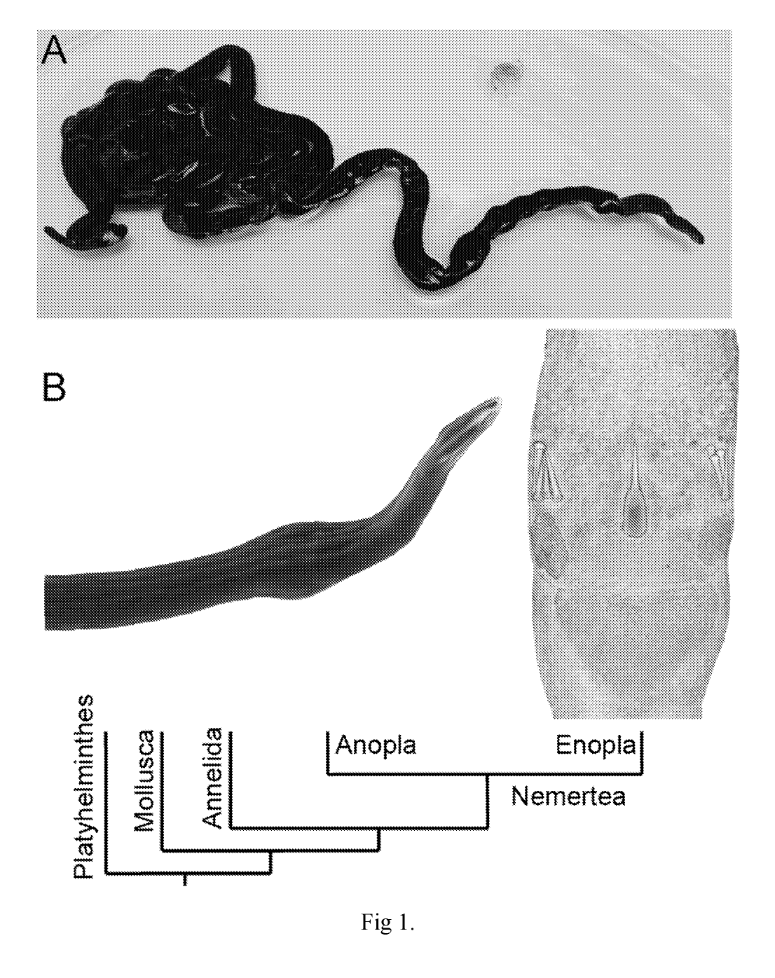

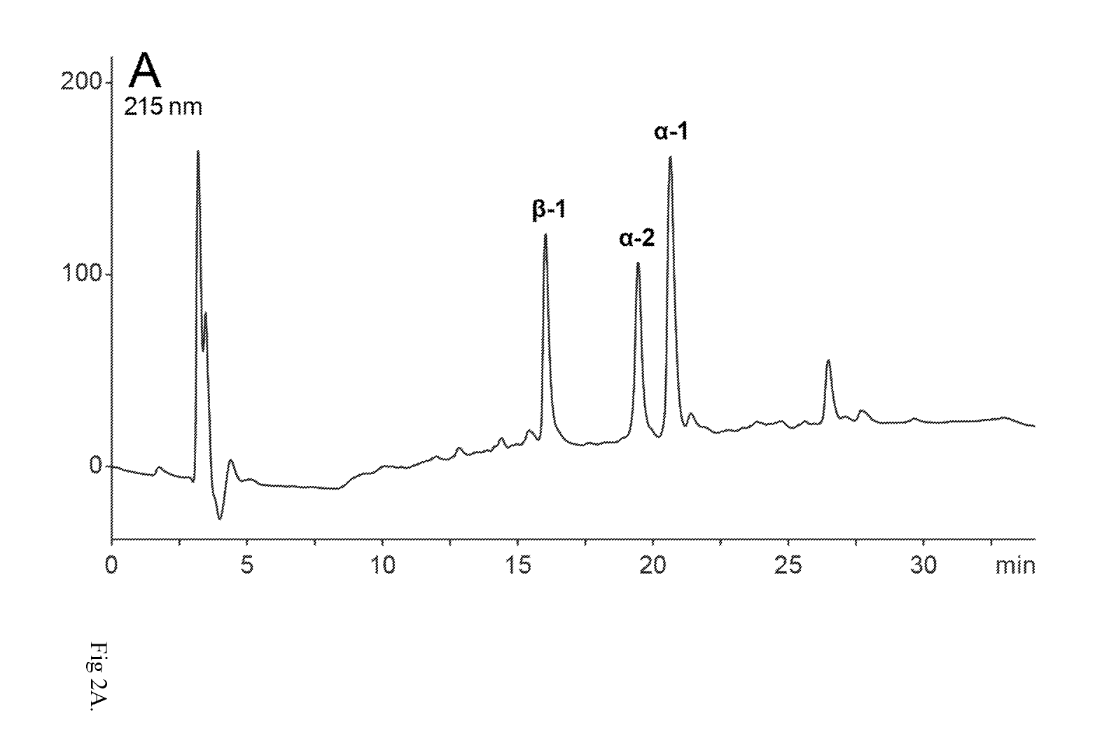

### SUMMARY OF THE INVENTION

The present invention relates to the following items. The subject matter disclosed in the items below should be regarded disclosed in the same manner as if the subject matter were disclosed in patent claims.
1. An isolated peptide or peptidomimetic, comprising an α-nemertide moiety, wherein:
   a) said α-nemertide moiety has a sequence according to SEQ ID NO:1; or
   b) said α-nemertide moiety has a sequence differing from SEQ ID NO:1 by residue substitutions, deletions or insertions numbering 1, 2, 3, 4, 5, 6, 7, 8 or 9 in total.
2. The peptide or peptidomimetic according to any of the preceding items, wherein said α-nemertide moiety has a sequence differing from SEQ ID NO:1 by residue deletions or insertions numbering 0, 1, 2, 3, 4, 5 or 6 in total.
3. The peptide or peptidomimetic according to item 1, wherein said α-nemertide moiety has a sequence differing from SEQ ID NO:1 by substitutions with alanine numbering 1, 2, 3, 4, 5, 6, 7, 8 or 9 in total.
4. The peptide or peptidomimetic according to item 1, wherein said α-nemertide moiety has a sequence differing from SEQ ID NO:1 by residue substitutions, deletions or insertions numbering 0, 1, 2, 3, 4, 5 or 6 in total.
5. The peptide or peptidomimetic according to any of the preceding items, wherein the α-nemertide moiety comprises six C residues at positions aligning with the positions of C residues in SEQ ID NO: 1.
6. The peptide or peptidomimetic according to any of the preceding items, wherein said α-nemertide moiety has a sequence differing from SEQ ID NO: 2 by residue substitutions, deletions or insertions numbering 1, 2, 3, 4, 5 or 6 in total.
7. The peptide or peptidomimetic according to any of the preceding items, wherein said α-nemertide moiety has a sequence differing from SEQ ID NO: 2 by alanine substitutions numbering 1, 2, 3, 4, 5 or 6 in total.
8. The peptide or peptidomimetic according to any of the preceding items, wherein said α-nemertide moiety has a sequence according to the consensus according to SEQ ID NO: 2.
9. The peptide or peptidomimetic according to any of the preceding items, wherein the α-nemertide moiety comprises a disulphide bridge between C residues located at positions aligning with the positions 2 and 16 of SEQ ID NO: 1.
10. The peptide or peptidomimetic according to any of the preceding items, wherein the α-nemertide moiety comprises a disulphide bridge between C residues located at positions aligning with the positions 9 and 20 of SEQ ID NO: 1.
11. The peptide or peptidomimetic according to any of the preceding items, wherein the α-nemertide moiety comprises a disulphide bridge between C residues located at positions aligning with the positions 15 and 26 of SEQ ID NO: 1.
12. The peptide or peptidomimetic according to any of the preceding items, wherein the α-nemertide moiety residue aligning with position 4 of SEQ ID NO: 1 is selected from the group consisting of A, K, S and P, preferably A.
13. The peptide or peptidomimetic according to any of the preceding items, wherein the α-nemertide moiety residue aligning with position 5 of SEQ ID NO: 1 is selected from the group consisting of T and V, preferably T.
14. The peptide or peptidomimetic according to any of the preceding items, wherein the α-nemertide moiety residue aligning with position 8 of SEQ ID NO: 1 is selected from the group consisting of F, V, G and M, preferably F.
15. The peptide or peptidomimetic according to any of the preceding items, wherein the α-nemertide moiety residue aligning with position 11 of SEQ ID NO: 1 is selected from the group consisting of L and I, preferably L.
16. The peptide or peptidomimetic according to any of the preceding items, wherein the α-nemertide moiety residue aligning with position 13 of SEQ ID NO: 1 is selected from the group consisting of N and K, preferably K.
17. The peptide or peptidomimetic according to any of the preceding items, wherein the α-nemertide moiety residue aligning with position 25 of SEQ ID NO: 1 is selected from the group consisting of K, H and A, preferably K.
18. The peptide or peptidomimetic according to any of the preceding items, wherein the α-nemertide moiety residue aligning with position 28 of SEQ ID NO: 1 is selected from the group consisting of P and K, preferably P.
19. The peptide or peptidomimetic according to any of the preceding items, wherein the α-nemertide moiety residue aligning with position 3 of SEQ ID NO: 1 is I.
20. The peptide or peptidomimetic according to any of the preceding items, wherein the α-nemertide moiety residue aligning with position 8 of SEQ ID NO: 1 is F.
21. The peptide or peptidomimetic according to any of the preceding items, wherein the α-nemertide moiety residue aligning with position 5 of SEQ ID NO: 1 is T.
22. The peptide or peptidomimetic according to any of the preceding items, wherein the α-nemertide moiety consists of a sequence according to SEQ ID NO:1 or any of SEQ ID NOs: 3-9.
23. The peptide or peptidomimetic according to any of the preceding items, wherein the α-nemertide moiety consists of the sequence according to SEQ ID NO:1.
24. The peptide or peptidomimetic according to any of the preceding items, wherein the α-nemertide moiety consists of the sequence according to SEQ ID NO:3.
25. The peptide or peptidomimetic according to any of the preceding items, wherein the α-nemertide moiety consists of a sequence according to SEQ ID NO:6.
26. The peptide or peptidomimetic according to any of the preceding items, wherein the peptide or peptidomimetic consists of a sequence according to SEQ ID NO:1.
27. The peptide or peptidomimetic according to any of the preceding items, wherein the peptide or peptidomimetic consists of a sequence according to SEQ ID NO:3.
28. The peptide or peptidomimetic according to any of the preceding items, wherein the sequence of the peptide or peptidomimetic consists of the sequence of the α-nemertide moiety.
29. The peptide or peptidomimetic according to any of the preceding items, wherein one or more the P residues in the α-nemertide moiety is/are hydroxylated.
30. The peptide or peptidomimetic according to any of the preceding items, wherein the α-nemertide moiety comprises a hydroxyproline at a position aligning with position 28 of SEQ ID NO: 1.
31. The peptide or peptidomimetic according to any of the preceding items, wherein the α-nemertide moiety comprises a hydroxyproline at a position aligning with position 29 of SEQ ID NO: 1.
32. The peptide or peptidomimetic according to any of the preceding items, wherein the α-nemertide moiety comprises a hydroxyproline at a position aligning with position 4 of SEQ ID NO: 1.
33. The peptide or peptidomimetic according to any of the preceding items, wherein the α-nemertide moiety contains no non-hydroxylated proline residues.
34. The peptide or peptidomimetic according to any of the preceding items, having a modified C-terminal, such as an amidated C-terminal.
35. The peptide or peptidomimetic according to any of the preceding items, having a modified N-terminal, such as an acylated N-terminal.
36. The peptide or peptidomimetic according to any of the preceding items, having a cyclic backbone.
37. The peptide or peptidomimetic according to any of the preceding items, comprising one or more non-natural residues.
38. The peptide or peptidomimetic according to any of the preceding items, comprising one or more D-amino acid residues.
39. The peptide or peptidomimetic according to any of the preceding items, comprising one or more non-peptide bonds in the backbone.
40. The peptide or peptidomimetic according to any of the preceding items, conjugated to a detectable marker, preferably biotin, a fluorescent marker, or a radioactive label.
41. The peptide or peptidomimetic according to any of the preceding items, wherein the peptide or peptidomimetic is a peptide having a sequence comprising at least one difference compared to any naturally occurring peptide sequence.
42. The peptide or peptidomimetic according to any of the preceding items, the peptide or peptidomimetic is a peptide having a sequence comprising at least one difference compared to any of the sequences according to SEQ ID NO:1 or any of SEQ ID NOs: 3-9.
43. The peptide or peptidomimetic according to any of the preceding items, wherein the peptide or peptidomimetic binds to a voltage-gated sodium channel.
44. The peptide or peptidomimetic according to any of the preceding items, wherein the peptide or peptidomimetic induces sustained non-inactivating currents on a voltage-gated sodium channel.
45. The peptide or peptidomimetic according to any of the preceding items, wherein the peptide or peptidomimetic induces sustained non-inactivating currents on a voltage-gated sodium channel with an EC50 of less than 5 μM, more preferably less than 0.5 μM, even more preferably less than 0.05 μM.
46. The peptide or peptidomimetic according to any of items 43-45, wherein the voltage-gated sodium channel is a vertebrate voltage-gated sodium channel.
47. The peptide or peptidomimetic according to item 46, wherein the vertebrate is a human, a rat or a mouse, preferably human.
48. The peptide or peptidomimetic according to any of items 43-45 wherein the voltage-gated sodium channel is a vertebrate voltage-gated sodium channels selected from human Naᵥ1.5, mouse Naᵥ1.6, rat Naᵥ1.4 and rat Naᵥ1.1.
49. The peptide or peptidomimetic according to any of items 43-45, wherein the voltage-gated sodium channel is an invertebrate voltage-gated sodium channel.
50. The peptide or peptidomimetic according to item 49 wherein the invertebrate voltage-gated sodium channel is selected from Blattella germanica Naᵥ1, Drosophila melanogaster Naᵥ1 and Verroa destructor Naᵥ1.
51. The peptide or peptidomimetic according to any of the preceding items, being a peptide.
52. The peptide or peptidomimetic according to any of items 1-50, being a peptidomimetic.
53. A method of manufacturing a peptide or peptidomimetic according to any of the preceding items, comprising:
   a. selecting a peptide or peptidomimetic structure according to any of items 1-52;
   b. synthesizing said peptide or peptidomimetic in vitro, preferably using solid phase peptide synthesis.
54. The method according to item 53, wherein the synthesis is carried out using Fmoc based solid phase peptide synthesis followed by oxidative folding.
55. A nucleic acid sequence, such as a DNA sequence, encoding a peptide according to any of items 1-52.
56. An expression vector comprising the nucleic acid sequence according to item 55, operably linked to a promoter.
57. A host cell comprising a nucleic acid sequence according to item 55 or a vector according to item 56.
58. A transgenic organism comprising a nucleic acid sequence according to item 55 or a vector according to item 56.
59. The transgenic organism according to item 58, wherein the organism is a plant.
60. The peptide or peptidomimetic according to any of items 1-52, for use as a medicament.
61. The peptide or peptidomimetic according to item 60, for use in the treatment of a condition selected from pain, neuropathic pain, diabetic pain, cancer pain, neuralgia, neuropathy, erythermalgia, osteoartrithis, cough and respiratory diseases connected to constriction of airways, for use as an anaesthetic or for use in blocking cough reflexes.
62. The peptide or peptidomimetic according to item 60, for use in the treatment or prevention of a parasitic infection, preferably a helminthic or ectoparasitic infection.
63. A use of the peptide or peptidomimetic according to any of items 1-52, as a pest control agent, preferably in agricultural pest control.
64. A use of the peptide or peptidomimetic according to any of items 1-52, as an insecticidal, molluscicidal or acaricidal agent.
65. A use of the peptide or peptidomimetic according to any of items 1-52, as an antihelmintic agent, preferably a nematicidal agent.
66. A use of the peptide or peptidomimetic according to any of items 1-52, as a voltage-gated sodium channel-binding reagent in an assay, preferably an in vitro assay.
67. A use of the peptide or peptidomimetic according to any of items 1-52, in an assay comprising determination of activity of a voltage-gated sodium channel.
68. A use of the peptide or peptidomimetic according to any of items 1-52, in an assay comprising determination of location of a voltage-gated sodium channel.

### DETAILED DESCRIPTION

The present invention discloses a novel class of peptides termed α-nemertides, based on the inventor's work on nemertide proteome and transcriptome as described in the appended Examples 1-6.

Peptide or Peptidomimetic

In a first aspect, there is provided an isolated peptide or peptidomimetic, comprising an α-nemertide moiety, wherein:
a) said α-nemertide moiety has a sequence according to SEQ ID NO:1; or
b) said α-nemertide moiety has a sequence differing from SEQ ID NO:1 by residue substitutions, deletions or insertions numbering 1, 2, 3, 4, 5, 6, 7, 8, 9, 10, 11, 12, 13, 14, 15 or 16 in total.

Preferably, the peptide or peptidomimetic of the first aspect may be a peptide. Alternatively, the peptide or peptidomimetic of the first aspect may be a peptidomimetic.

The α-nemertide moiety may be part of a larger peptide or peptidomimetic, or the peptide or peptidomimetic may consist of the α-nemertide moiety.

Said α-nemertide moiety may have a sequence differing from SEQ ID NO:1 by residue deletions or insertions numbering 0, 1, 2, 3, 4, 5 or 6 in total.

Said α-nemertide moiety may have a sequence differing from SEQ ID NO:1 by substitutions with alanine numbering 1, 2, 3, 4, 5, 6, 7, 8 or 9 in total.

Said α-nemertide moiety may have a sequence differing from SEQ ID NO:1 by residue substitutions, deletions or insertions numbering 0, 1, 2, 3, 4, 5 or 6 in total.

The α-nemertide moiety may comprise six C residues at positions aligning with the positions of C residues in SEQ ID NO: 1. Without being bound by theory, it is noted often be the case that C-residues are among the most conserved residues between homologues.

Said α-nemertide moiety may have a sequence differing from the consensus sequence according to SEQ ID NO: 2 by residue substitutions, deletions or insertions numbering 1, 2, 3, 4, 5 or 6 in total.

Said α-nemertide moiety may have a sequence differing from the consensus sequence (SEQ ID NO: 2) by alanine substitutions numbering 1, 2, 3, 4, 5 or 6 in total.

Preferably, said α-nemertide moiety has a sequence according to the consensus sequence of SEQ ID NO: 2.

Preferably, the sequence of the peptide or peptidomimetic consists, or essentially consists of the sequence of the α-nemertide moiety. The sequence may have 0, 1, 2, 3, 4, 5, 6, 7, 8, 9 or 10 residues in addition to the sequence of the α-nemertide moiety, preferably 0.

Disulfide Bridges

The α-nemertide moiety may comprise a disulphide bridge between C residues located at positions aligning with the positions 2 and 16 of SEQ ID NO: 1. The α-nemertide moiety may comprise a disulphide bridge between C residues located at positions aligning with the positions 9 and 20 of SEQ ID NO: 1. The α-nemertide moiety may comprise disulphide bridges between C residues located at positions aligning with the positions 15 and 26 of SEQ ID NO: 1. Preferably, all of the disulphide bridges mentioned above are present in the peptide or peptidomimetic. The aforementioned arrangement of disulphide bridges corresponds to the naturally occurring α-nemertide of Lineus longissimus, as shown in Example 1. As shown in Example 6, reduction of the disulphide bridges results in significantly lowered activity.

Preferred Sequence Features

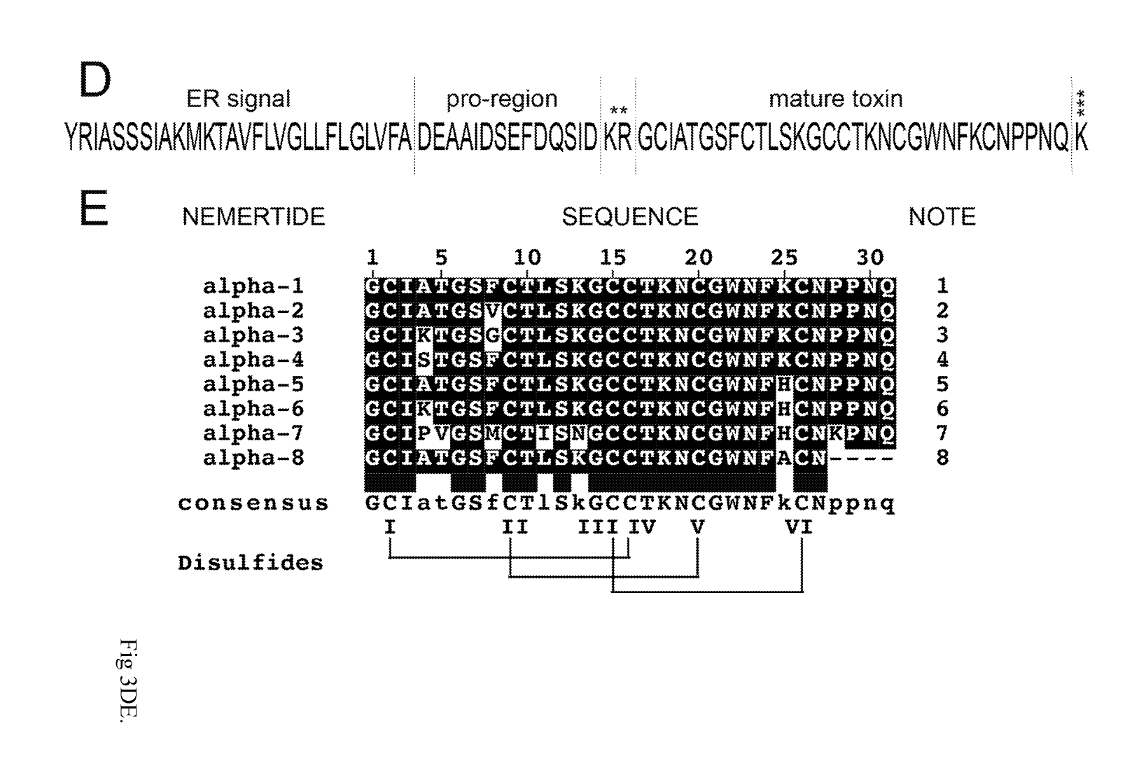

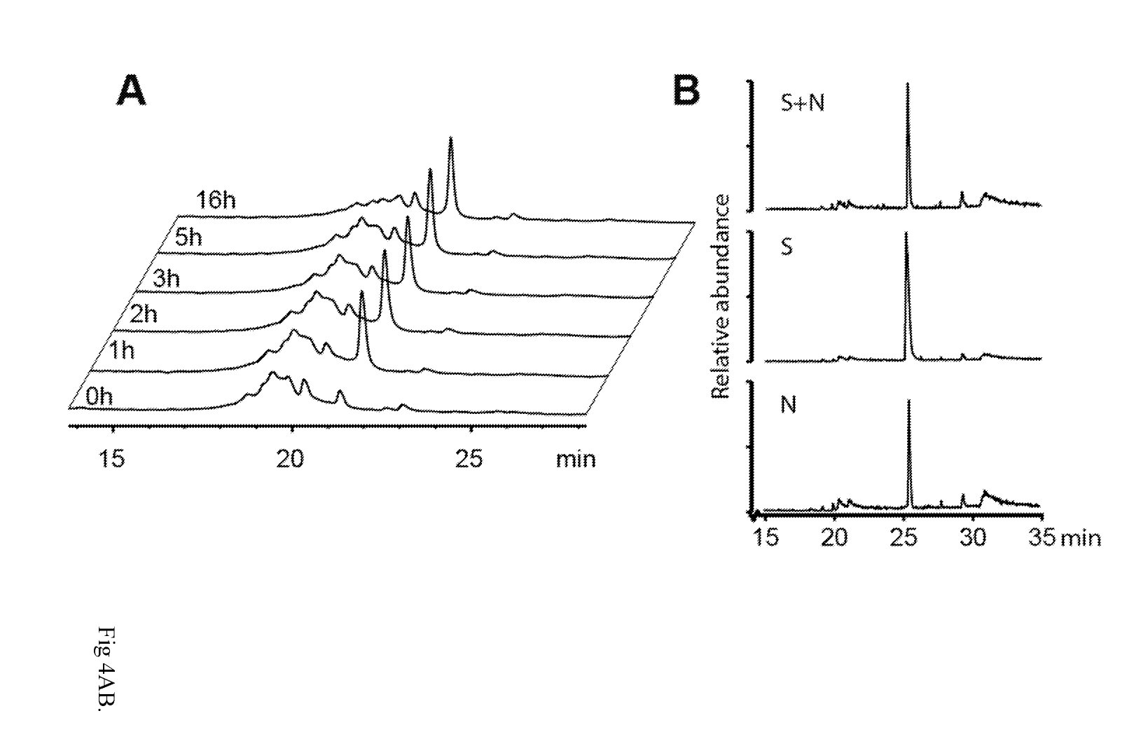

Based on sequence alignment between α-nemertides from various species (see FIG. 3E), as well as the comparative experiments presented as Examples 5 and 6, certain residues at certain positions are deemed particularly preferable, as detailed below.

The α-nemertide moiety residues aligning with positions 4 and 8 of SEQ ID NO: 1 may be hydrophobic, for example A and F, respectively.

The α-nemertide moiety residue aligning with position 4 of SEQ ID NO: 1 may be selected from the group consisting of A, K, S and P, preferably A.

The α-nemertide moiety residue aligning with position 5 of SEQ ID NO: 1 may be selected from the group consisting of T and V, preferably T.

The α-nemertide moiety residue aligning with position 8 of SEQ ID NO: 1 may be selected from the group consisting of F, V, G and M, preferably F.

The α-nemertide moiety residue aligning with position 11 of SEQ ID NO: 1 may be selected from the group consisting of L and I, preferably L.

The α-nemertide moiety residue aligning with position 13 of SEQ ID NO: 1 may be selected from the group consisting of N and K, preferably K.

The α-nemertide moiety residue aligning with position 25 of SEQ ID NO: 1 may be selected from the group consisting of K, H and A, preferably K.

The α-nemertide moiety residue aligning with position 28 of SEQ ID NO: 1 may be selected from the group consisting of P and K, preferably P.

The α-nemertide moiety residue aligning with position 3 of SEQ ID NO: 1 is preferably I.

The α-nemertide moiety may consist of a sequence according to SEQ ID NO:1 or any of SEQ ID NOs: 3-9, preferably SEQ ID NO:1 (α1), SEQ ID NO:3 (α2) or SEQ ID NO: 6 (α5), most preferably SEQ ID NO: 6 (α5).

The peptide or peptidomimetic may consist of a sequence according to SEQ ID NO:1 or any of SEQ ID NOs: 3-9, preferably SEQ ID NO:1 (α1), SEQ ID NO:3 (α2) or SEQ ID NO: 6 (α5), most preferably SEQ ID NO: 6 (α5)

Proline Hydroxylations

As shown in Example 1, the prolines of native L. longissimus α-nemertides are post-translationally modified into hydroxyprolines. Thus, one or more the P residues in the α-nemertide moiety of the first aspect are preferably hydroxylated.

Preferably, the α-nemertide moiety comprises a hydroxyproline at a position aligning with position 28 of SEQ ID NO: 1, at a position aligning with position 29 of SEQ ID NO: 1, and/or at a position aligning with position 4 of SEQ ID NO: 1. Most preferably, the α-nemertide moiety contains no non-hydroxylated proline residues.

Non-Natural Features

Various modifications to peptides and peptidomimetics, in order to modify and improve the properties of the peptide are within reach of the skilled person based on the teachings herein, and are therefore regarded as being within the scope of the present invention. The following relates to a number of preferable modifications, but it is understood that many other modifications are also possible within the scope of the claims of the present invention.

The peptide or peptidomimetic may have a modified C-terminal or N-terminal, such as an amidated C-terminal or an acylated N-terminal.

The peptide or peptidomimetic may have a cyclic backbone.

The peptide or peptidomimetic may comprise one or more non-natural residues.

The peptide or peptidomimetic may comprise one or more D-amino acid residues.

The peptide or peptidomimetic may comprise one or more non-natural bonds in the backbone.

The peptide or peptidomimetic may be conjugated to a detectable marker, preferably biotin, a fluorescent marker or a radioactive label.

The sequence of the peptide or peptidomimetic may comprise at least one difference compared to any naturally occurring peptide sequence. Preferably, the sequence of the peptide or peptidomimetic comprises at least one difference compared to any of the sequences according to SEQ ID NO:1 or SEQ ID NOs: 3-9.

Functional Features

The peptide or peptidomimetic of the first aspect may have the property of binding to a voltage-gated sodium channel, preferably in a selective manner.

The peptide or peptidomimetic may have the property of inducing sustained non-inactivating currents on a voltage-gated sodium channel.

Preferably, the induction is demonstrated in in vivo models with an EC50 of less than 5 μM, more preferably less than 1 μM, even more preferably less than 0.1 μM, determined using the methodology demonstrated in Example 2 using crustaceans. The EC50 determination may also be done in insects or vertebrates such as fish. The EC50 may also be determined by brine shrimp-killing effect in an Artemia salina assay (see Examples 5 and 6), where the EC50 may be less than 10 μM, preferably less than 10 μM, more preferably less than 1 μM, most preferably less than 0.3 μM.

Preferably, the induction is achieved on voltage gated sodium channel receptors expressed in oocytes measured using patch clamp technology with an EC50 of less than 5 μM, more preferably less than 0.5 μM, even more preferably less than 0.05 μM. The EC50 may be determined using the methodology of Example 3.

Said voltage-gated sodium channel may be a vertebrate voltage-gated sodium channel, preferably a human, a rat or a mouse voltage-gated sodium channel, most preferably human.

The voltage-gated sodium channel may be a vertebrate voltage-gated sodium channels selected from human Naᵥ1.5, mouse Naᵥ1.6, rat Naᵥ1.4 and rat Naᵥ1.1.

The voltage-gated sodium channel may alternatively be an invertebrate voltage-gated sodium channel, preferably selected from Blattella germanica NaV1, Drosophila melanogaster NaV1 and Varroa destructor NaV1.

Method of Manufacture

In a second aspect, there is provided a method of manufacturing a peptide or peptidomimetic according to any of the preceding claims, comprising:
a. selecting a peptide or peptidomimetic having structure or sequence in accordance with the first aspect;
b. synthesizing said peptide or peptidomimetic in vitro.

Assembly of the peptide chain may be carried out using solid phase peptide synthesis (SPPS), preferably Fmoc based solid phase peptide synthesis. SPPS synthesis is followed by oxidative folding of the fully reduced peptide, preferably in a solution containing a mixture of reduced and oxidised glutathione (e.g. 2 and 4 mM respectively).

Assembly of the peptide chain and may also be carried out by recombinant expression, preferably in a bacterium (e.g. E. coli), a fungus (e.g. yeast) or a plant. Oxidative folding may be done in vivo, using the aforementioned expression system, or in vitro as described above.

Transgenic Aspects

In a third aspect, the present invention provides a nucleic acid sequence (such as DNA, RNA, or the like) encoding a peptide of the first aspect of the present invention.

In a fourth aspect, there is provided an expression vector comprising the nucleic acid sequence according to the third aspect, operably linked to a promoter.

In a fifth aspect, there is provided a host cell comprising a nucleic acid sequence according to the third aspect or a vector according to the fourth aspect.

In a sixth aspect, there is provided a transgenic organism comprising a nucleic acid sequence according to the third aspect or a vector according to the fourth aspect. Preferably, the transgenic organism is a bacterium, a fungus or a plant. Plants engineered to express a peptide of the first aspect would produce a peptide toxic to invertebrates feeding on the plant thus conferring resistance to pests, in particular insects and helminths.

Medical Uses

In a seventh aspect, there is provided a peptide or peptidomimetic according to the first aspect, for use as a medicament. In other words, there is provided a method of treatment for a disease, comprising administering a peptide or peptidomimetic according to the first aspect to a subject in need thereof. Furthermore, there is provided a use of a peptide or peptidomimetic according to the first aspect, in the manufacture of a medicament.

In an eighth aspect, the peptide or peptidomimetic according to the first aspect may be for use in the treatment of a condition selected from pain, neuropathic pain, diabetic pain, cancer pain, neuralgia, neuropathy, erythermalgia, osteoartrithis, cough and respiratory diseases connected to constriction of airways, and for use as an anaesthetic or for use in blocking cough reflexes.

The peptide or peptidomimetic according to the first aspect may also be for use in the treatment or prevention of a parasitic infection or infestation, preferably a helminthiasis or an infection or infestation by ectoparasites.

The helminthiasis may be selected from:
a) Infection by a soil-transmitted helminth, including Ascaris lumbricoides, Trichuris trichiura, Necator americanus, Strongyloides stercoralis and Ancylostoma duodenale, Hymenolepis nana, Taenia saginata, Enterobius spp., Fasciola hepatica, Schistosoma mansoni, Toxocara canis, Toxocara cati,
b) Infection by roundworms (nematodiasis) including Filariasis (Wuchereria bancrofti, Brugia malayi infection), Onchocerciasis (Onchocerca volvulus infection), Trichostrongyliasis (Trichostrongylus spp. infection), Dracunculiasis (guinea worm infection),
c) Infection by tapeworms (cestodiasis), including Echinococcosis (Echinococcus infection), Hymenolepiasis (Hymenolepis infection), Taeniasis/cysticercosis (Taenia infection), Coenurosis (T. multiceps, T. serialis, T. glomerata, and T. brauni infection),
d) Infection by trematodes (trematodiasis) including Amphistomiasis (Amphistomes infection), Clonorchiasis (Clonorchis sinensis infection), Fascioliasis (Fasciola infection), Fasciolopsiasis (Fasciolopsis buski infection), Opisthorchiasis (Opisthorchis infection), Paragonimiasis (Paragonimus infection), Schistosomiasis/bilharziasis (Schistosoma infection), and
e) Infection by Acanthocephala including Moniliformis infection.

The ectoparasite infection or infestation may be selected from lice such as crab louse (pubic lice) or pediculosis (head lice), Lernaeocera branchialis-infection (cod worm), linguatulosis, porocephaliasis, fleas, ticks or a mite infection such as scabies.

For the treatment, the peptide or peptidomimetic may be administered in a suitable composition to the infected subject in a manner bringing the peptide or peptidomimetic in contact with the parasite being treated. For prevention, the peptide or peptidomimetic is administered to a subject at risk for acquiring a parasitic infection, in a manner bringing the peptide or peptidomimetic in contact with body parts typically affected by said potential parasites, or body parts typically used by the parasites to gain entry to the body.

The peptide or peptidomimetic may administered in any suitable manner including systemically, enterally, parenterally or topically. For the treatment of ectoparasites, the preferred mode of administration is topical to the site of infection, e.g. skin, mucous membrane or hair. For topical administration, the peptide or peptidomimetic may be in a composition formulated as a cream, salve, powder, ointment, gel, liquid or the like.

In other words, there is provided a method of treatment for a condition selected from the aforementioned list, or aforementioned use, comprising administering a peptide or peptidomimetic according to the first aspect to a subject in need thereof. Furthermore, there is provided a use of a peptide or peptidomimetic according to the first aspect in the manufacture of a medicament for a condition selected from the aforementioned lists, or aforementioned uses.

Pest Control Uses

In a ninth aspect, there is provided a use of the peptide or peptidomimetic according to the first aspect, in pest control, in particular within agriculture, forestry, horticulture, managed turf and lawns, and building protection. Also provided is a method, comprising administering a peptide or peptidomimetic according to the first aspect to pests or their environment.

In a tenth aspect, there is provided a use of the peptide or peptidomimetic according to the first aspect, as an insecticidal, molluscicidal or acaricidal agent. Also provided is a method, comprising administering a peptide or peptidomimetic according to the first aspect to an insect, a mollusc, an arachnid or its environment.

In an eleventh aspect, there is provided a use of the peptide or peptidomimetic according to the first aspect, as an antihelmintic agent, for example a nematicidal agent. Also provided is a method, comprising administering a peptide or peptidomimetic according to the first aspect to a helminth or its environment.

Preferably, such administration is done by formulating the peptide or peptidomimetic agent into a composition provided in a variety of physical forms, e.g. baits, sprays, gels, powders, impregnated films, granules, or liquids and applying the composition in concentrated or diluted form to the pest in question or to their environment, for example, to plants or trees, soil, seeds, stored crops, and building materials, at a time and in a manner so as to act prophylactically and/or therapeutically. Said pest may be among others include nuisance, disease and damage pests such as cockroaches, mosquitos and mites, and plant pests such as arthropods including beetles, locusts and grasshoppers, lepidoptera, flies, true bugs, thrips, aphids, nematodes, always understood to include all life stages thereof.

Research Uses

In a twelfth aspect, there is provided a use of the peptide or peptidomimetic according to the first aspect, as a voltage-gated sodium channel-binding ligand as a research tool in an assay, to study the pharmacological or physiological role of ion channel activity, or as a marker compound to display ion channel binding sites. Also provided is a method, comprising contacting a peptide or peptidomimetic according to the first aspect with a voltage-gated sodium channel, and determining the degree of binding.

In a thirteenth aspect, there is provided a use of the peptide or peptidomimetic according to the first aspect, in an assay comprising determination of activity of a voltage-gated sodium channel. Also provided is a method, comprising contacting peptide or peptidomimetic according to the first aspect with a voltage-gated sodium channel, and determining the sodium channel activity.

The specificity of binding of the peptide or peptidomimetic allows it to be used as a probe to locate its specific targets, for example voltage gated sodium channels in samples, such as on tissue sections in an in vivo imaging assay. In a fourteenth aspect, there is provided a use of the peptide or peptidomimetic according to the first aspect, in an assay comprising determination of location of a voltage-gated sodium channel. Also provided is a method, comprising contacting peptide or peptidomimetic according to the first aspect with a sample comprising voltage-gated sodium channels under conditions allowing binding of the peptide or peptidomimetic to the voltage-gated sodium channels, followed by determining location of the peptide or peptidomimetic. In this application, it is particularly preferable that the peptide or peptidomimetic be labelled with a detectable marker, such as a fluorescent marker or a radioactive label.

The assay of the twelfth, thirteenth or fourteenth aspect may be an in vitro assay, or an in vivo assay.

Further Details Concerning the Present Invention

In the present application, the inventors disclose a novel family of peptide toxins from nemerteans, and describe their structure and activity. Peptides were discovered in the mucus and epidermis of the Lineus longissimus. This novel family, which was named the α-nemertides, appears to be limited to the genus Lineus, as judged by data mining of a series of nemertean transcriptomes that have become available recently (Romiguer J et al. Nature 2014, 515, 261-3; Andrade S C S et al. Mol. Biol. Evol. 2014, 31, 3206-15; Whelan N V et al. Genome Biol. Evol. 2015, 6, 3314-25).

Whereas bioinformatics helped to discover α-nemertides, the key to the discovery was the combination with biochemical analyses: the extraction of peptides and analyses using LC-MS and MALDI-imaging. It is clear that it was the use of the peptidomic approach and that the need of detailed analyses at the peptide level increases with the number of posttranslational modifications and with a decreasing sequence length.

According to the present disclosure, the family of α-nemertides comprises at least seven, 31-amino acid residues long, peptides. The discovered peptides contain three disulfides arranged in an inhibitory cystine knot (ICK) motif. The solution structure of α-1 reveals a compact fold, with the N-terminal stabilized by the Cys2-Cys16 disulfide. The C-terminal appears to be more flexible, and contains two Hyp residues. The two α-nemertides (α-1 and α-2) isolated from Lineus longissiumus differ only with a Phe to Val substitution at position 8; the Phe is a part of a hydrophobic patch together with Phe 22 and Trp 28. In the family of α-nemertides, residues 4, 8 and 25 are subjects of variations (4, AKSP; 8, FVGM; 25, KHA): all these positions are displayed at the same side of the molecule. It is not unlikely that these structural variations control preference between ion channel types or subtypes, and in the present invention the importance of the hydrophobic residues in positions 4 and 8 is demonstrated. Single mutations at these positions is shown to confer different activities in the range of orders of magnitudes. The nature of the ribbon worm, its use of a proboscis—in some cases armed with a stiletto, in combination with the ICK motif and the size of the peptides, suggest a parallel to cone snails and their toxins. However, differences are substantial: on the molecular level, sequences of α-1 to α-7 have no homology to any other peptide or protein. In addition, whereas cone snail venoms contain complex libraries of hundreds to thousands of different peptides (Biass D, Violette A, Hulo N, Lisacek F, Favreau P and Stocklin R J. Proteome Res., 2015, 14 (2), pp 628-638), Lineus longissimus apparently only express two α-nemertides judged on peptide and RNA level. Similarly, other species that were analysed for the presence of alpha nemertides in the present application showed the presence of only two or three peptides also (Table 2).

TABLE 2
Occurrence of α-nemertides in the species studied.

| Species | α-1 | α-2 | α-3 | α-4 | α-5 | α-6 | α-7 | α-8 |
|---|---|---|---|---|---|---|---|---|
| Lineus lacteus | X | | X | | | | | |
| Lineus | X | X | | | | | | |
| longissimus | | | | | | | | |
| Lineus | | | X | | X | | | |
| pseudolacteus | | | | | | | | |
| Lineus ruber | X | X | | | | | X | |
| Lineus | | | | X | | X | | |
| sanguinensis | | | | | | | | |
| Riseriellus | | | | | | | | X |
| occultus | | | | | | | | |

In Lineus longissimus, α-1 and α-2, appear in similar amounts as judged by HPLC-UV, which may suggest that they are equally important but act on different targets. As described herein, the activity in the brine shrimp assay differs by one order of magnitude between these two peptides in that assay, which supports that theory. Interestingly, this pattern is repeated for other species in the same assay: all appear to express one peptide that are more potent in this particular assay.

However, a sequence resembling the proposed peptide maturation enzyme tex-31 from Conus textile was found in the L. longissimus transcriptome. Tex-31 cleaves a conotoxin propeptide with two basic residues in P1 and P2 positions, and preferably a leucine in P4 position; its presence and likely a similar processing site on the N-terminal side of mature α-1 nemertides suggest a possible common initial posttranslational processing pathway.

The closest match to the three-dimensional structure of α-1 is the human liver expressed antimicrobial peptide-2, LEAP-2 followed by two ICK spider toxins, ω-HXTX-Hv1a (previously; ω-ACTX-Hv1a) and κ-TRTX-Scg1a (previously; SGTx1). Backbone similarity of LEAP-2 and α-1 is striking, but LEAP-2 contains two disulfides only and the pattern of surface hydrophobicity does not overlap. The physiological function of LEAP-2 is not clear, despite its name, but the homologous spider toxins are targeting voltage-gated calcium channels (ω-HXTX-Hv1a) and voltage-activated potassium channels (κ-TRTX-Scg1a).

So what is the function and target of the nemertides? It is not apparent if are they used for capture of prey or for defense, or for both? It is clear though, that the large amount of mucus that is released by Lineus longissimus when challenged by a (physical) threat contains substantial amounts of nemertides α-1, α-2, and beta-1; and the effect that the mucus exerts is obvious by the numbing feeling of the skin experienced when holding the worm in the palm of your hand.

Crustaceans are well known preys and possible predators of nemerteans, and both lobsters (Homarus americanus) and green crabs (Carcinus maenas) have been used to assay activity in vivo of nemertean chemistry. In the current study, the activity of nemertide α-1 was characterized in detail on crabs, revealing immediate neurotoxic activity at a dose of 1 μg/kg, and death within minutes at 10 μg/kg. This can be compared with the activity of neurotoxin B-IV (paralytic dose: 2.1 μg/kg, lethal dose: 23 μg/kg) (Kem W R. J. Biol. Chem. 1976, 251, 4184-92) tetrodotoxin (lethal dose 10 ug/kg).

Nemertide α-1 exerts its neurotoxic activity by slowing down the inactivation of Naᵥ channels. The α-1 induced alteration of steady-state inactivation most likely results from the toxin binding to site 3. Many α-scorpion toxins, spider and sea anemone toxins are known to bind to this site; on binding, they trap the voltage-sensor S4 of DIV in its inward or deactivated position, hereby preventing the structural movements required for fast inactivation (Stevens M et al. Front. Pharmacol. 2, 71). Several toxins capable of binding site 3 have been isolated from marine organisms, mainly sea anemones. The insect-specificity of α-1 is demonstrated by the complete inhibition of the inactivation of the insect Naᵥ channels DmNav1 and BgNa1, and it is furthermore emphasized by the 100-fold (0.8 μM to 8 nM) difference in EC₅₀ values between mammalian (Nav1.6) and insect (DmNav1) channels.

The preference for invertebrate Naᵥ channels and the potency in vivo in arthropods suggests a potential use of nemertide α-1 as insecticide, or as a lead compound in the development of novel insecticides. Spider toxins suggested suitable as insecticidal leads have immediate neurotoxic effects in the range of 10-100 pmol/g when injected intrathoracically; in comparison, the effect of α-1 at 1 μg/kg equals an amount of ^{˜}300 femtomol/g. Although the number of nemertide toxins appears limited compared to toxins from other phyla, they represent novel sequences and structures. Most likely, each of these nemertides has different Naᵥ channel selectivity: this explains why α-1 and α-2 occur in similar abundance in the mucus.

General Aspects Relevant to Present Disclosure

The term “comprising” is to be interpreted as including, but not being limited to. All references are hereby incorporated by reference. The arrangement of the present disclosure into sections with headings and subheadings is merely to improve legibility and is not to be interpreted limiting in any way. In particular, the division does not in any way preclude or limit combining features under different headings and subheadings with each other. The scope of patent protection is solely determined by the appended claims.

### EXAMPLES

The following examples are not to be regarded as limiting. For further information on the experimental details, the skilled reader is directed to a separate section titled Materials and Methods.

### Example 1: Discovery of Nemertides

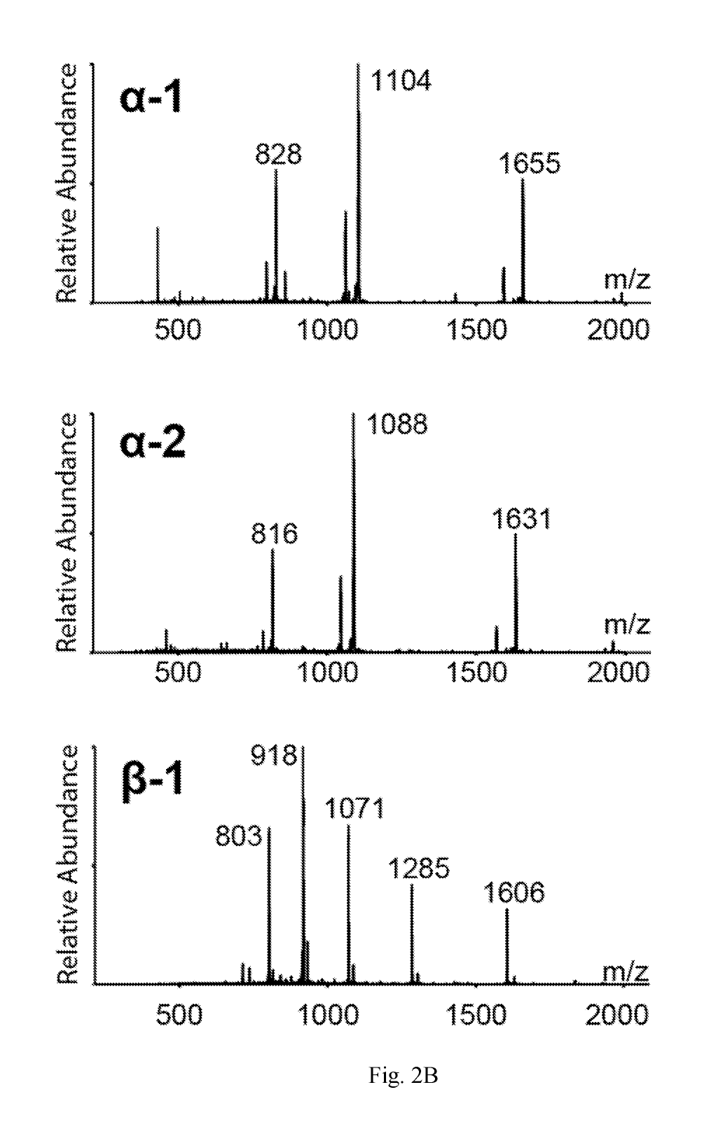

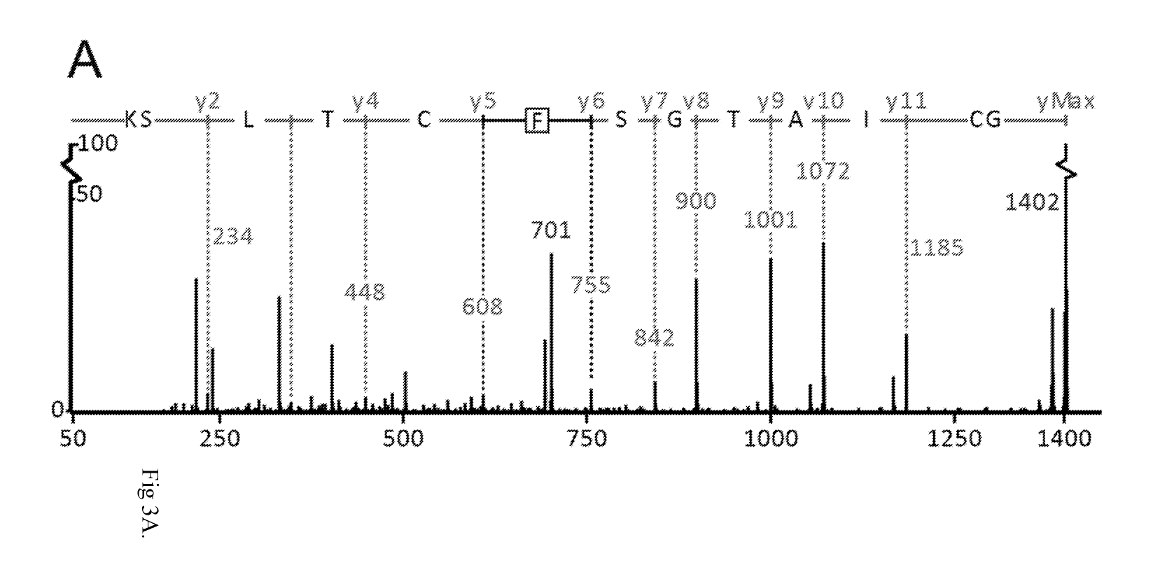

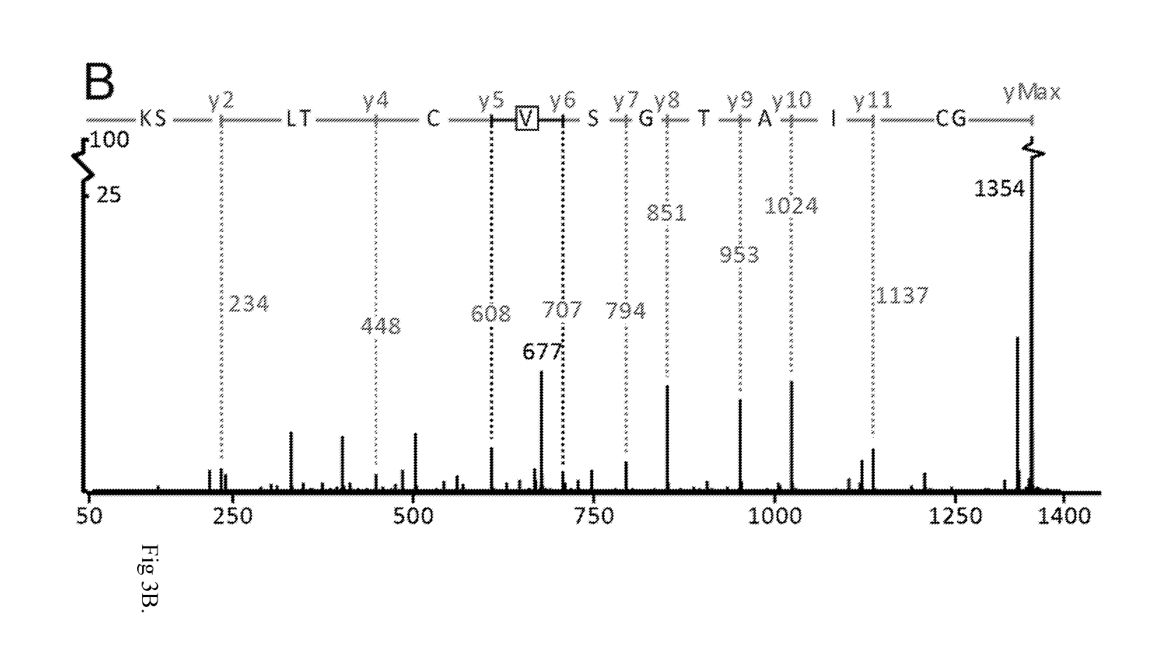

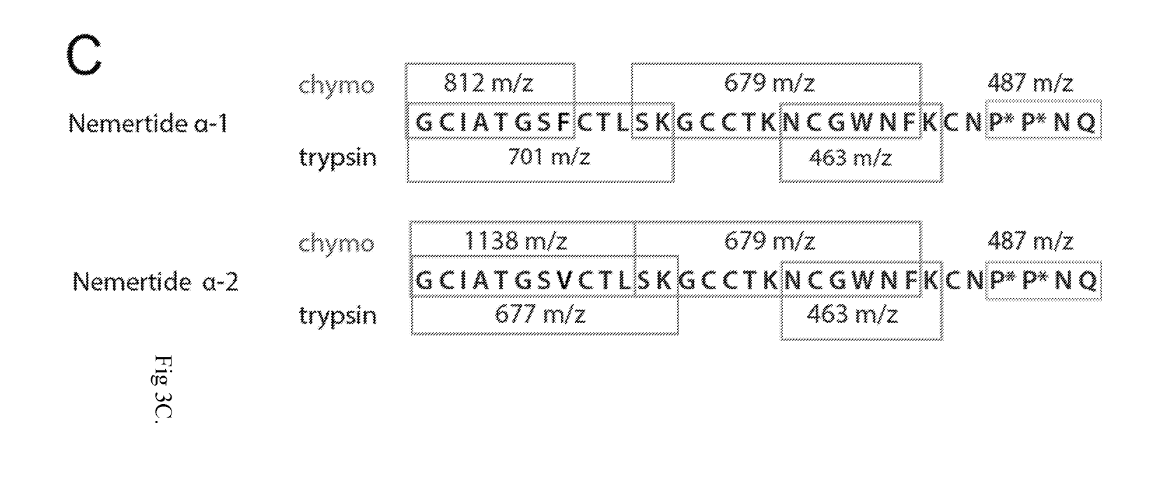

The mucus covering the body of Lineus longissimus was collected, lyophilized and resuspended in a solvent suitable for LC-MS to investigate the chemistry of toxins. Having previously established that no TTX is present in the mucus, attention was turned to compounds of higher molecular weights. The analyses revealed three prominent peaks with deconvoluted masses (M+H⁺) of 3308.35, 3260.40 (mo) and 6419.00 (av.). Subsequently, these compounds were isolated in μ-grams amounts using a combination of gel filtration and RP-HPLC. MALDI imaging demonstrates that peptide occurrence is limited to the epidermis and to the mucus layer. Results from transversal cuts across the mid-section of one specimen is shown in FIG. 2C: all peptides show the same distribution.

Isolated peptides were reduced and alkylated using iodoacetamide, which increased the molecular weights by 348 Da for the two smaller compounds and 464 Da for the larger one. These increments in mass correspond to the presence of three and four disulfide bonds, respectively. Combined with the relatively small difference in mass (Δ 47.95) between the two smaller compounds, these results indicated the occurrence of two classes of peptides in the mucus. Quantitative amino acid analyses supported this indication, and we grouped and named these peptides accordingly: the 3 kDa peptides are called nemertide α-1 and α-2, respectively, and the larger peptide nemertide β-1. For the α-class, experimental masses from analyses by MS differed from masses calculated from the net composition from amino acid analyses by 32 Da, suggesting further posttranslational modifications.

Alkylated peptides were subjected to enzymatic digestion to generate peptide fragments amenable for LC-MSMS sequencing, using trypsin, chymotrypsin and endoproteinase Glu-C, in separate experiments. Some fragments of α-1 and α-2 showed identical masses and retention times, demonstrating homology between peptides. Two of these, m/z 463²⁺ and 679²⁺, revealed identical 14-residue long sequences. Other fragments differed between peptides, including two ions with Δ 47.95: the tryptic 701²⁺ fragment of α-1 and 677²⁺ of α-2. MSMS sequencing of these fragments showed that these peptide fragments differ by a Phe to Val substitution (Δ −48.00), as shown in FIGS. 3A and B. In total, MSMS sequencing revealed 25 out of 31 residues of α-1 and α-2. The sequence was not determined for β-1. We then turned to transcriptome sequencing to determine the missing parts of the peptides.

The transcriptome of Lineus longissimus was sequenced using a combined pool of RNA isolated from transversal dissections along the body of a single specimen on an Illumina HiSeq2000. The assembled transcriptome contains 81597 contigs, with a total length of 91,851,747 bp. tBLASTn searches using the sequences determined by MSMS as queries suggested the full length sequence of α-1, which show a good fit to MS data if the prolines are hydroxylated. The difference of 32 Da between the net composition of amino acids and the molecular weight of the peptide or peptidomimetic may thus be explained by the presence of hydroxyprolines (Hyp). In addition, the sequence of nemertide β-1 could be determined with the help of the transcriptome; it is homologous to neurotoxin BIV as previously reported by Blumenthal (Blumenthal K M et al. J. Biol. Chem. 1981, 256, 9063-7): peptides comprise 57 and 55 residues respectively, four disulfide bonds and two hydroxyprolines. The sequence of nemertide α-2 could not be determined conclusively from our transcriptome, but could be identified in the transcriptome published recently by Whelan and coworkers (Whelan N V et al. Genome Biol. Evol. 2014, 6, 3314-25).

BLAST searches of 17 nemertean transcriptomes sequenced so far, reveled homologues to both α- and β-nemertides. The number of identified sequences are limited: only seven complete α-nemertide sequences were identified. In particular, the α-class of toxins seems to be very well conserved in the Linean linage (genera Lineus and Riseriellus) with representatives in all sampled species. A partial sequence belonging to the α-nemertides could also be found in the transcriptome of R. occultus. The small differences found in the α-toxins were mostly present in the N-terminal part, with the highest variation in position 4 (residues: AKSP) and 8 (residues: FVGM). Also position 25 has some variation (residues: KHA). 77% of the sequences overlap, when the probably partial α-8 (R. occultus) was removed.

No homologue to α-1 or α-2 sequences was found in any other database. We then decided to characterize this novel class of peptides in detail, using nemertide α-1 as the prototype.

### Example 2: Nemertide α-1 is a Potent Toxin to Crustaceans

Having the sequence of the α-nemertides in hand, we turned to solid phase peptide synthesis (SPPS) to provide material enough for biological and structural characterization. Nemertide α-1 was assembled using FMOC-chemistry on an HMPA-resin. The use of a combination of manual and microwave assisted SPPS resulted in good yield of crude peptide (92%). The presence of the two Hyp-residues was unambiguously confirmed by comparing retention times and MS/MSMS spectra of reduced, and reduced and alkylated, peptides with and without modified Pro-residues.

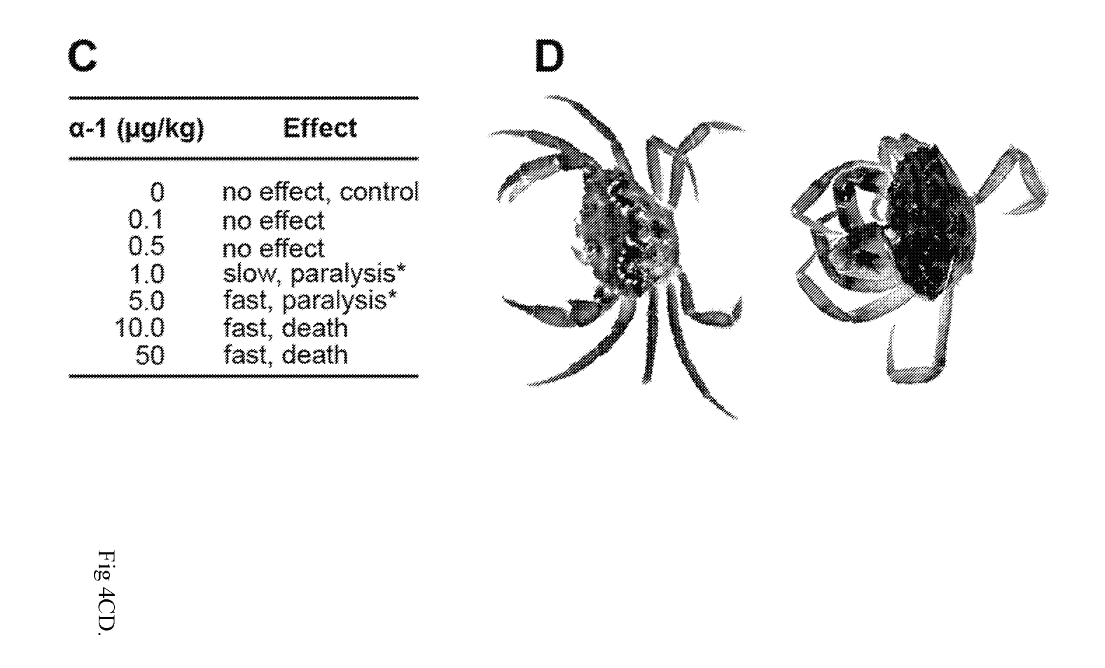

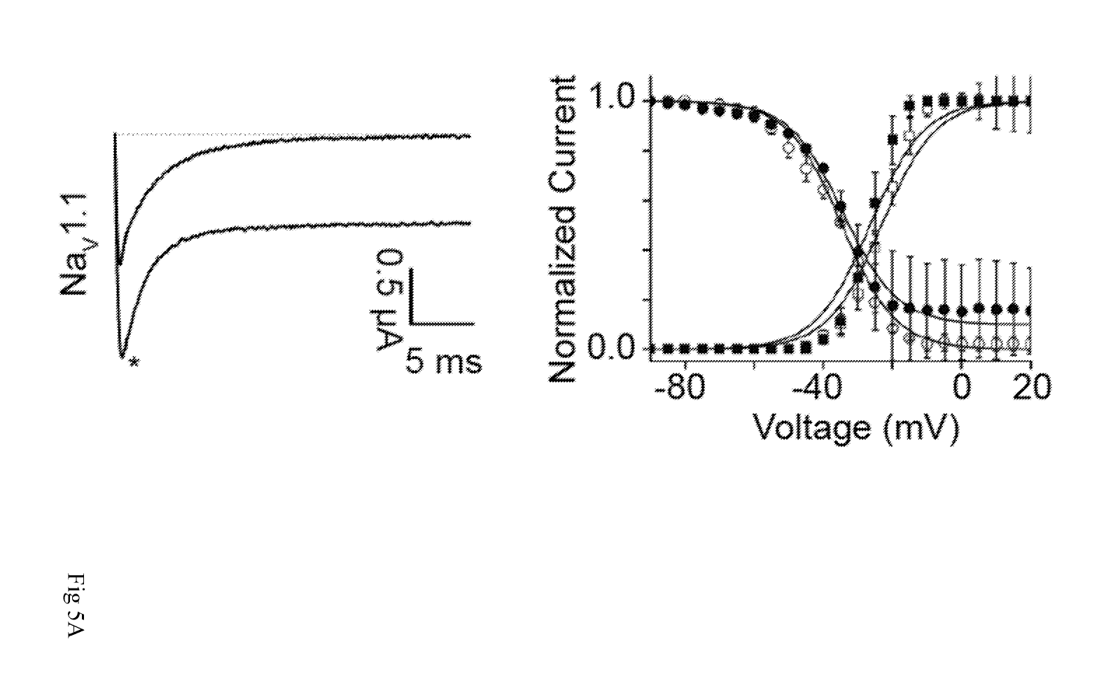

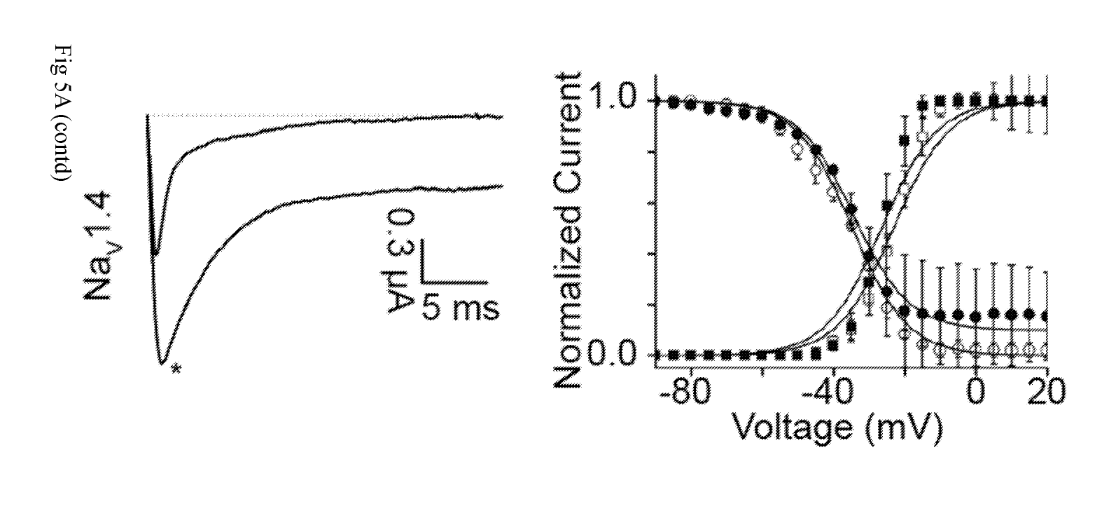

Crude peptide was directly subjected to oxidative folding in 2 mM 0.4 mM reduced and oxidized glutathione, 0.1 M ammonium hydrogen bicarbonate and 20% isopropanol, as shown in FIG. 4A. The final yield of peptide was 9%. Synthetic and native α-1 was mixed and co-injected on two different separation systems: identical retention times and mass demonstrate conformity of these peptides.

The effect of nemertean mucus on crustaceans is well documented, and the shore crab has been used as a model system to assess biological activity in vivo. When injected with active concentrations of nemertide α-1, tremor of the limbs was seen within 1-2 minutes. This was followed by hypertonus, and claws and legs were pulled inwards the body and the crab tilted forward. From this point the crabs remained in a paralytic state. After 20-30 minutes hypertonus was released while paralysis remained. No recovery could be observed for any dose showing effect: the higher doses proved lethal, whereas low doses brought on a later onset partial paralysis. This was the case for the lowest dose to provoke an effect (1 μg/kg), whereas a rapid response, as outlined above, was observed for high-dose injections (5-50 μg/kg).

### Example 3: Nemertide α-1 Preferentially Targets Invertebrate Naᵥs Compared to Vertebrate Naᵥs

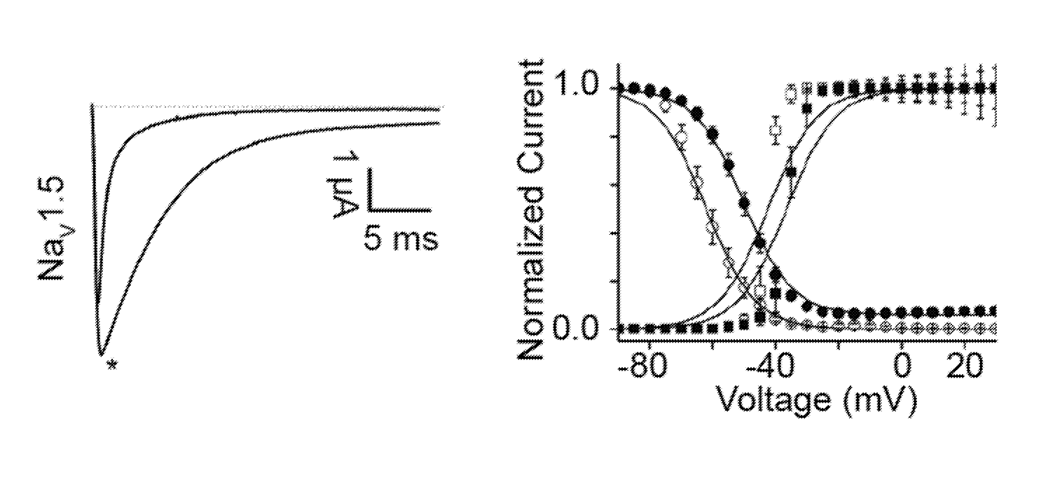

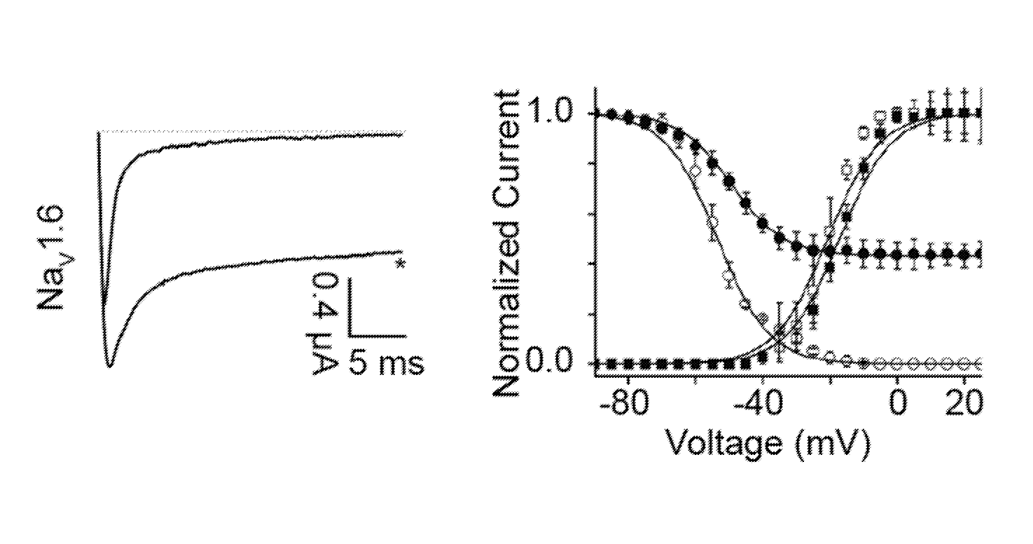

Nemertide α-1 was investigated for its activity on five vertebrate and three invertebrate voltage-gated sodium channel isoforms (Navs). For the mammalian Navs, a concentration of 6 μM nemertide α-1 significantly delayed the inactivation of Nav1.1, Nav1.4, Nav1.5 and Nav1.6 (FIG. 5A). No effect was seen on Nav1.8. Steady-state activation and inactivation curves were constructed to characterize the modulation of Nav channels upon toxin binding. No significant alterations in the kinetics of gating were observed for Nav1.1 channels. The midpoint of activation for Nav1.4 did not shift significantly but the V1/2 of inactivation shifted from −38.1±0.2 mV in control to −48.6±0.4 mV in the presence of α-1. For Nav1.6, the V1/2 values of activation were −20.8±0.1 mV in the control and −17.3±0.3 mV after application of 6 μM α-1. Similarly, the V1/2 of inactivation were shifted from −53, 6±0.5 mV to −49.8±0.2 mV in control and toxin conditions respectively. Both the activation and inactivation curves of Nav1.5 channels were altered. The midpoint of activation shifted from −42.4±0.1 mV in control to −36.3±0.1 mV after toxin application while for the inactivation curve values of −62.2±0.2 mV in control and −50.6±0.1 mV in the presence of toxin were observed. To assess the concentration dependence of the α-1-induced modulatory effects on Nav1.6 channels, a concentration-response curve was constructed (FIG. 5B). The EC50 value was determined to 0.8±0.1 μM.

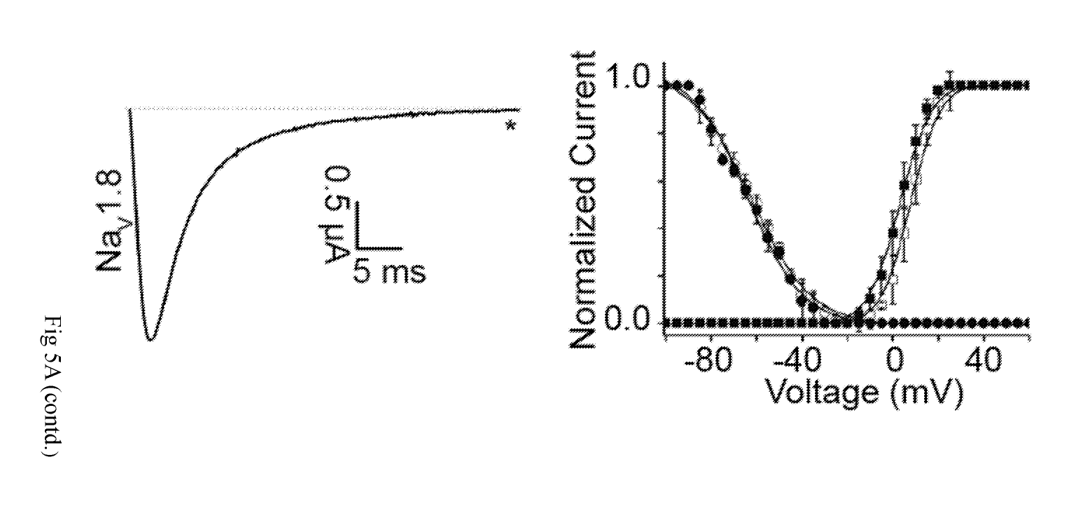

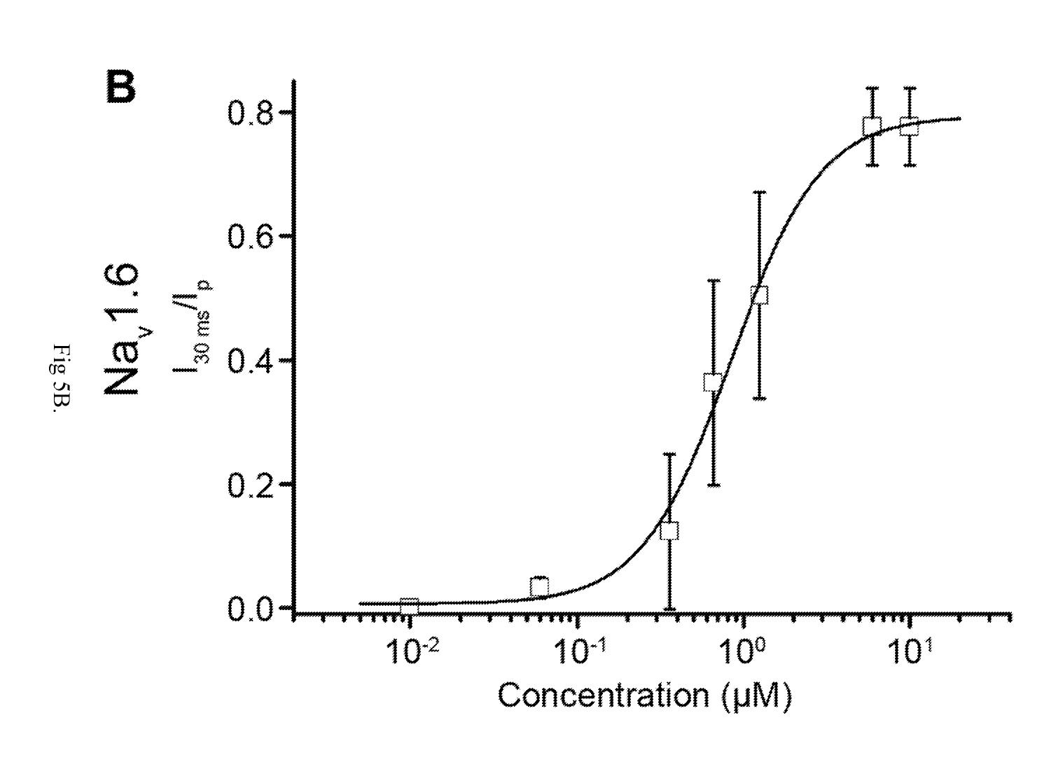

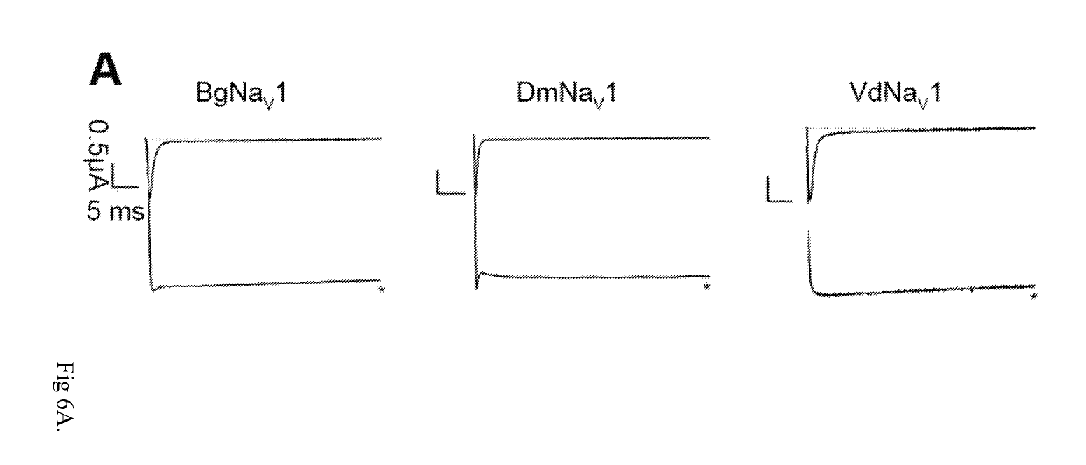

α-1 demonstrated a profound effect on the inactivation of invertebrate Naᵥ channels of Blattella germanica (BgNaᵥ1), Drosophila melanogaster (DmNaᵥ1) and Verroa destructor (VdNaᵥ1) (FIG. 6). At 1 μM α-1 completely inhibited their inactivation, resulting in sustained non-inactivating currents. BgNav1 channels were used to further characterize the mechanism of α-1 activity. α-1 acts in a concentration dependent way with a EC50 value of 8.6±2.9 nM (FIG. 6).

No significant alteration of activation was noted: V_{1/2} values were determined to −29.1±2.2 mV in the control and −32.5±3.2 mV after addition of 10 nM α-1 (FIG. 6). For the inactivation curves, V_{1/2} shifted from −60.4±0.6 mV in the control to −54.5±1.6 mV with 10 nM α-1, respectively. α-1 significantly enhanced the recovery from inactivation. τ values yielded 11.6±0.4 ms and 3.8±0.5 ms in control and after application of 10 nM α-1, respectively (FIG. 6). To investigate the state-dependence of inhibition, the following protocol was used. As control, a series of depolarizing pulses was applied to an oocyte expressing BgNaᵥ channels. Thereafter, 10 nM α-1 was added and no pulsing was performed for 2 minutes. Next, a similar series of pulses was executed. A strong degree of delay of inactivation was observed after the 2 minutes incubation, indicating that the open state is not required for toxin interaction with the channel (FIG. 6). FIG. 6 shows current traces evoked by a 50 ms depolarization from −90 mV to −30 mV in control and after application of 10 nM α-1. A massive increase in sodium influx can be noted after toxin modulation of the channel kinetics of gating (FIG. 6).

### Example 4: α-Nemertides Define a New Family of Toxins

The three-dimensional structure of α-1 was determined using solution NMR. Homonuclear and heteronuclear two dimensional spectra were collected for sequential peak assignments using standard methods (Wutrich K, NMR of Proteins and Nucleic Acids, 1986, Wiley), TOCSY and NOESY spectra are found as Supplementary Information. Overall, spectra were of excellent quality with well dispersed signals indicating a defined structure. Structures were calculated after determination of inter-proton distance and dihedral angle restraints, and hydrogen bonds identified from a temperature gradient experiment.

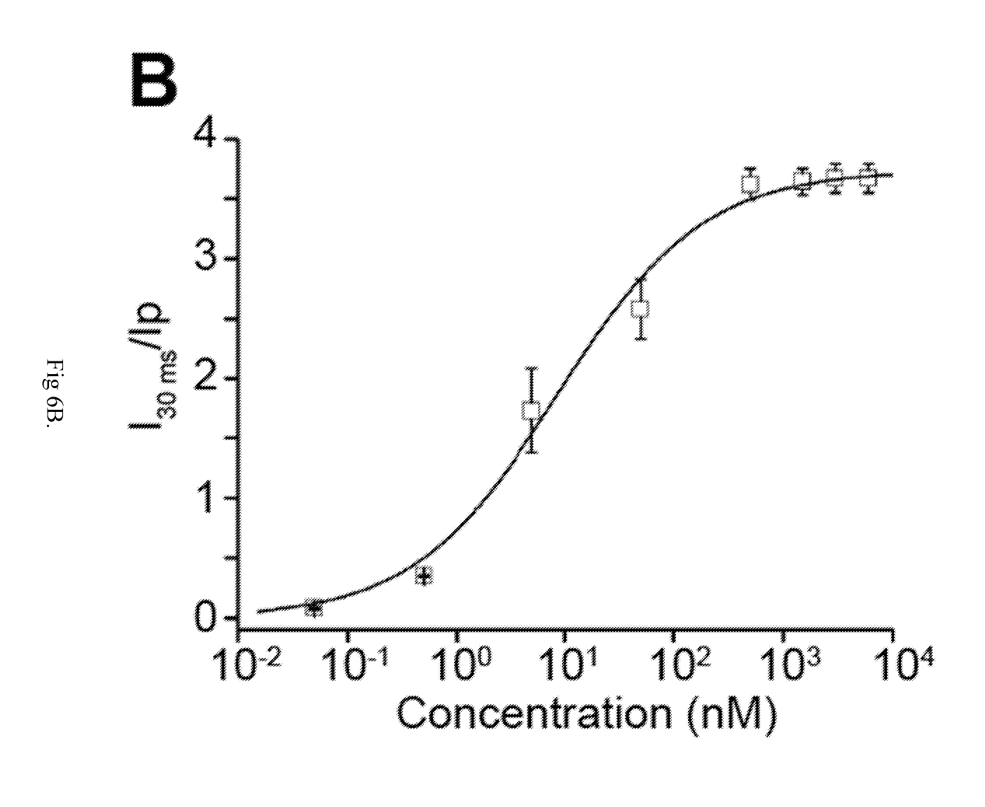

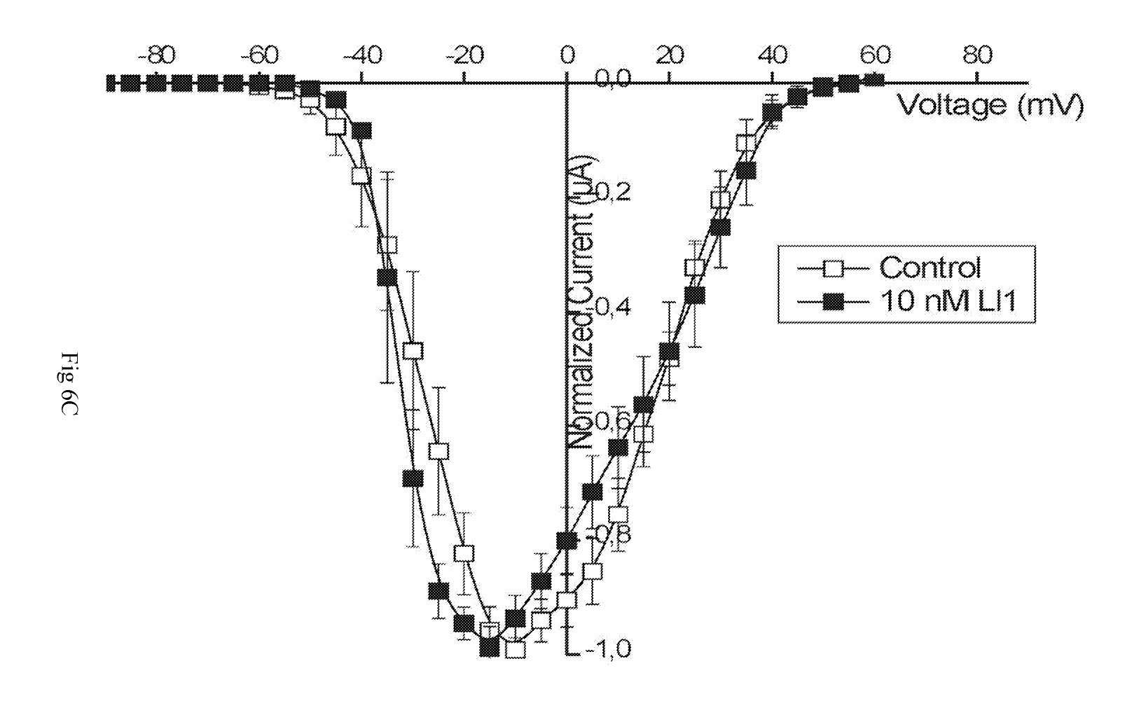

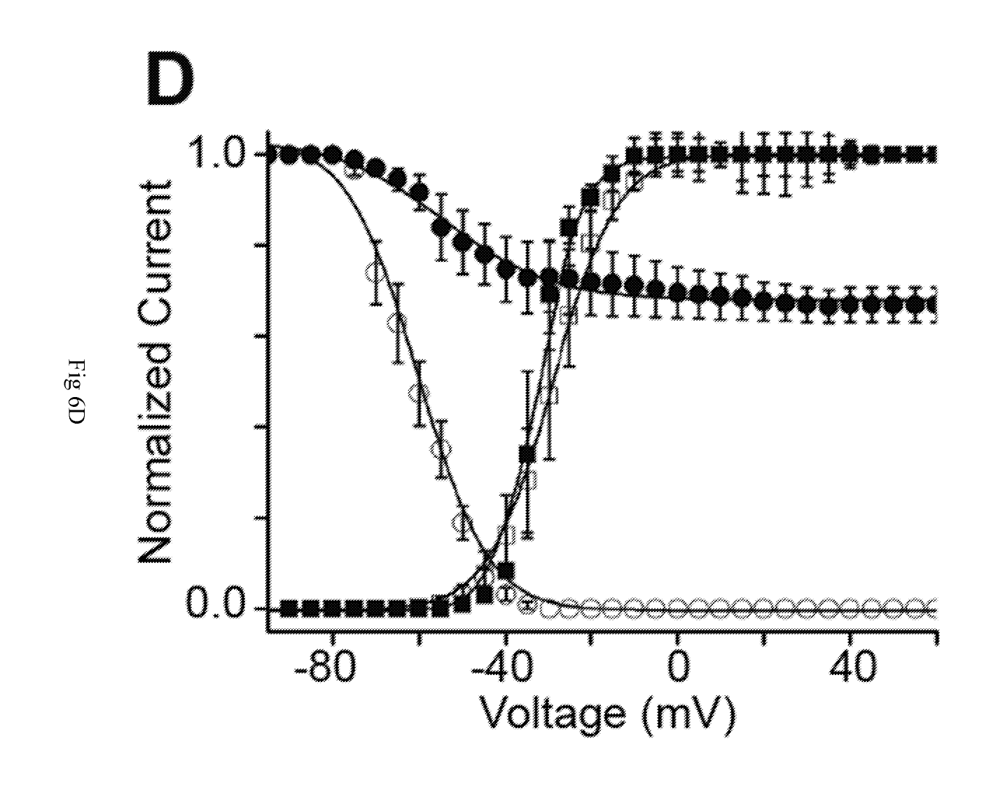

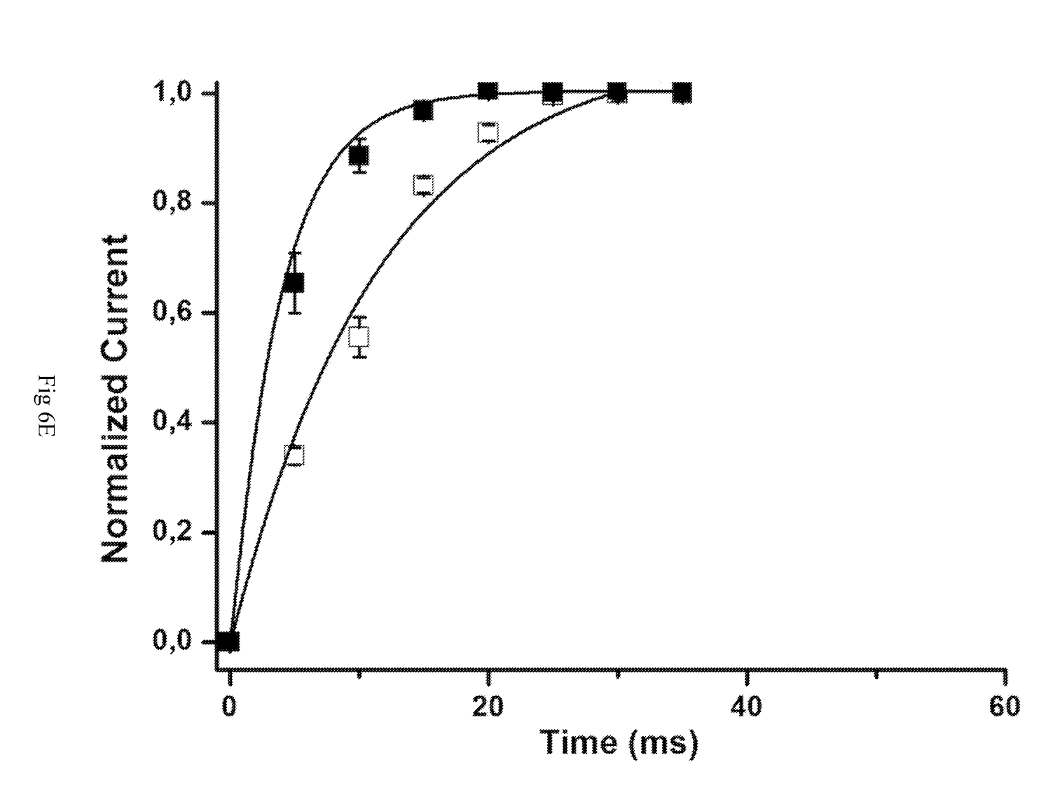

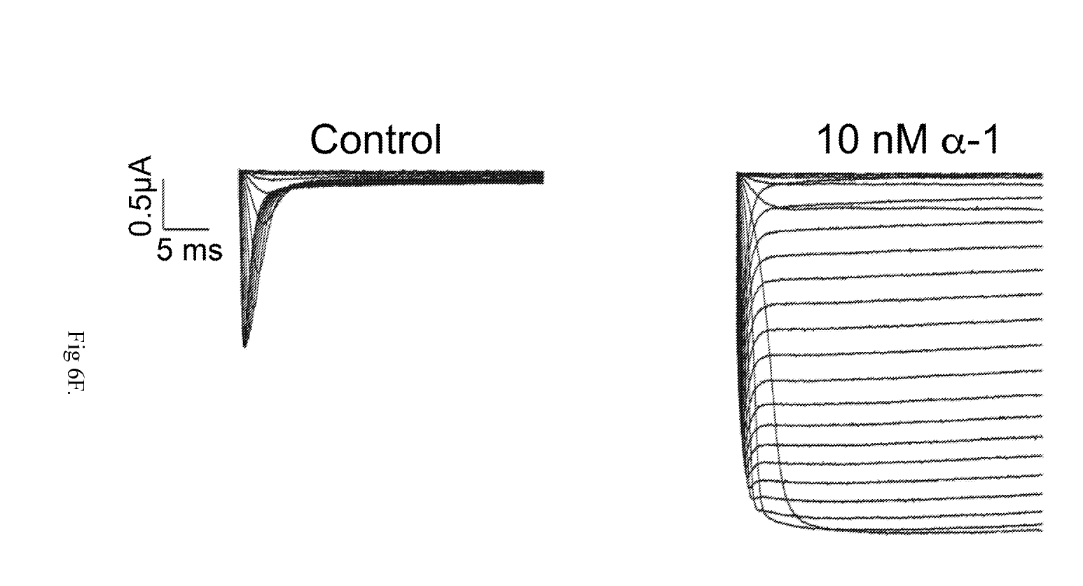

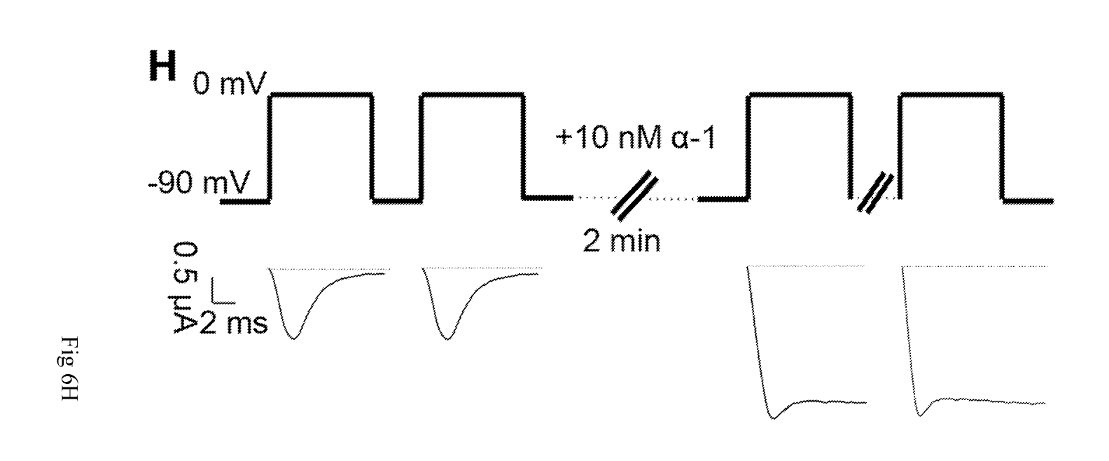

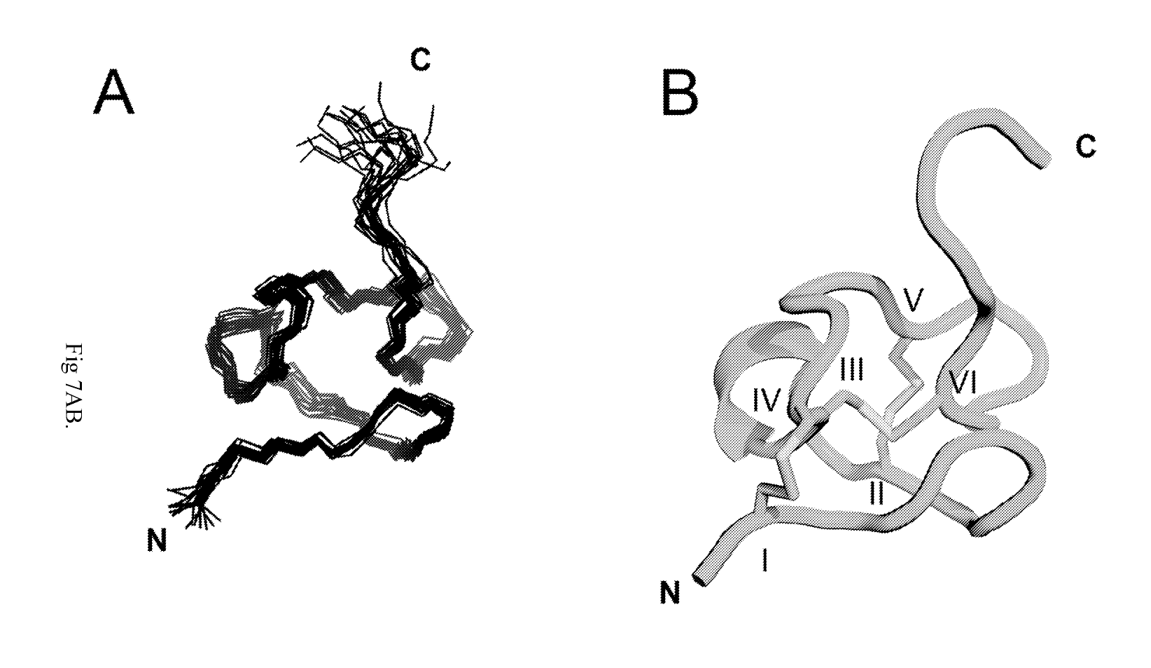

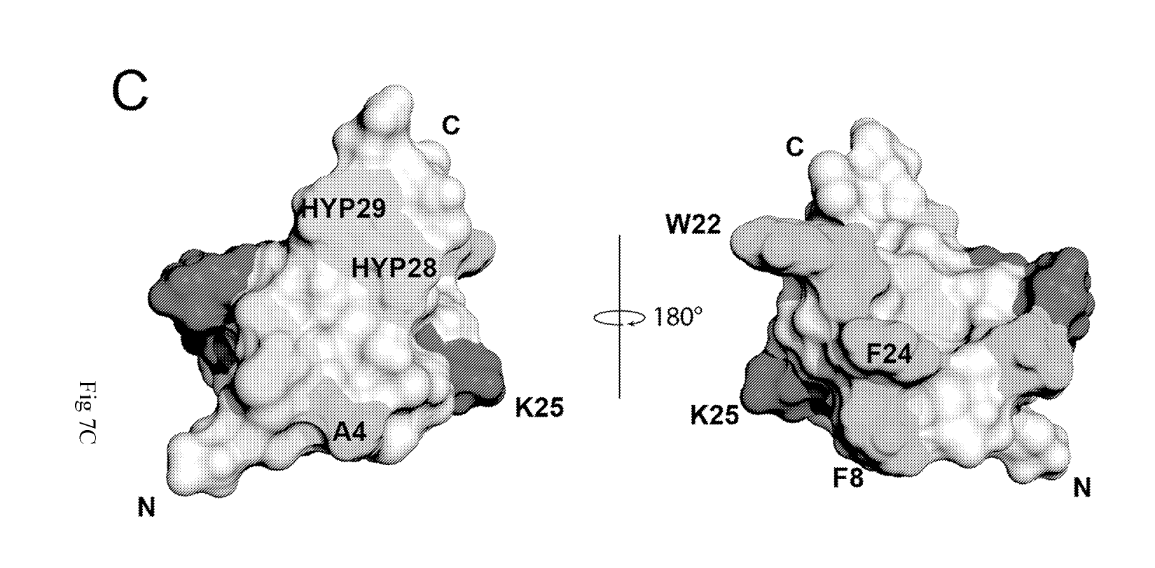

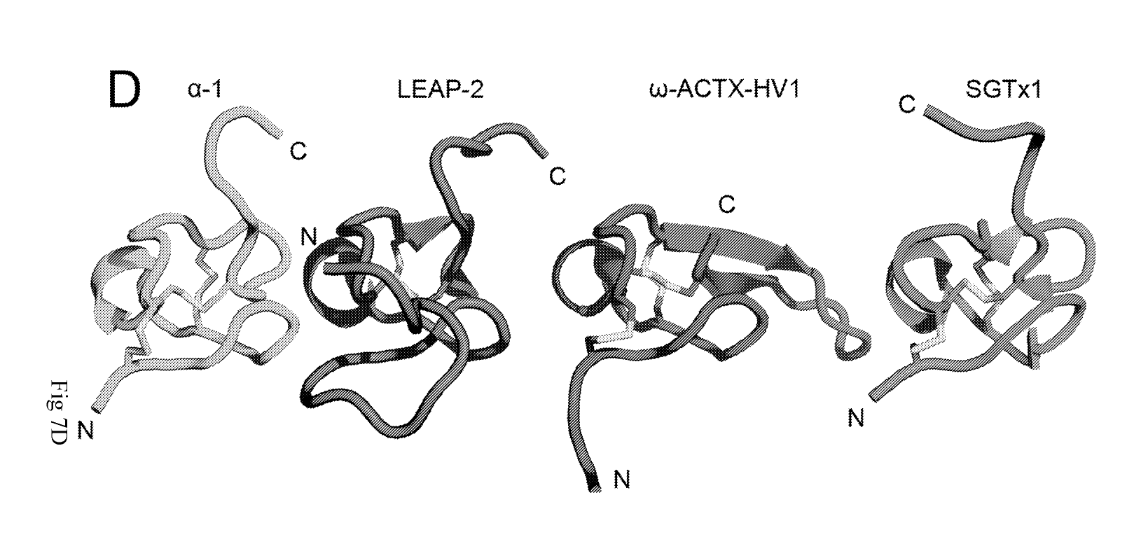

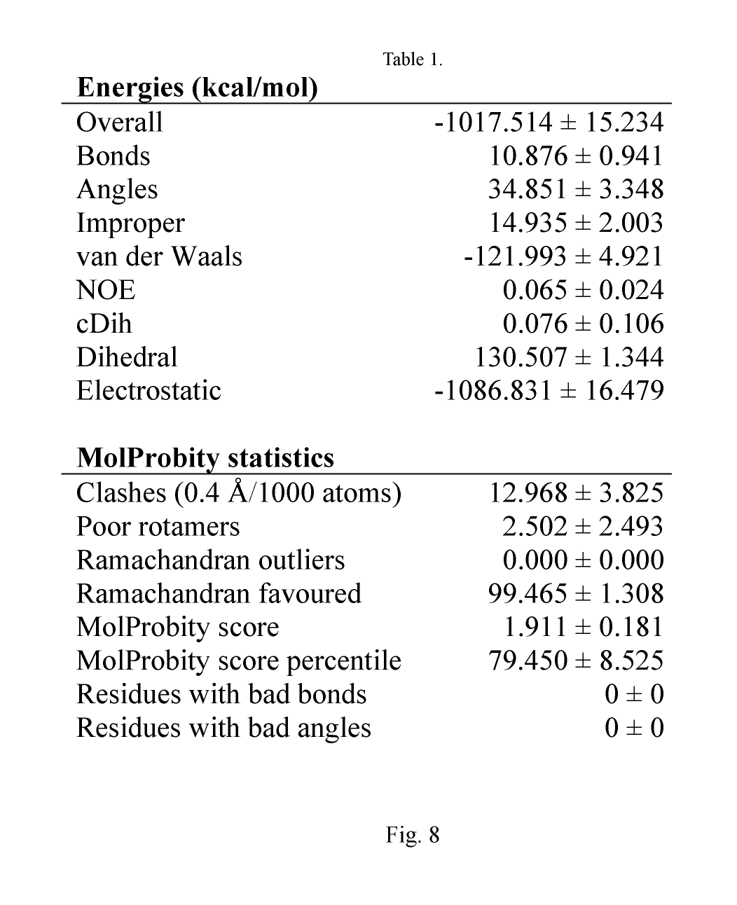

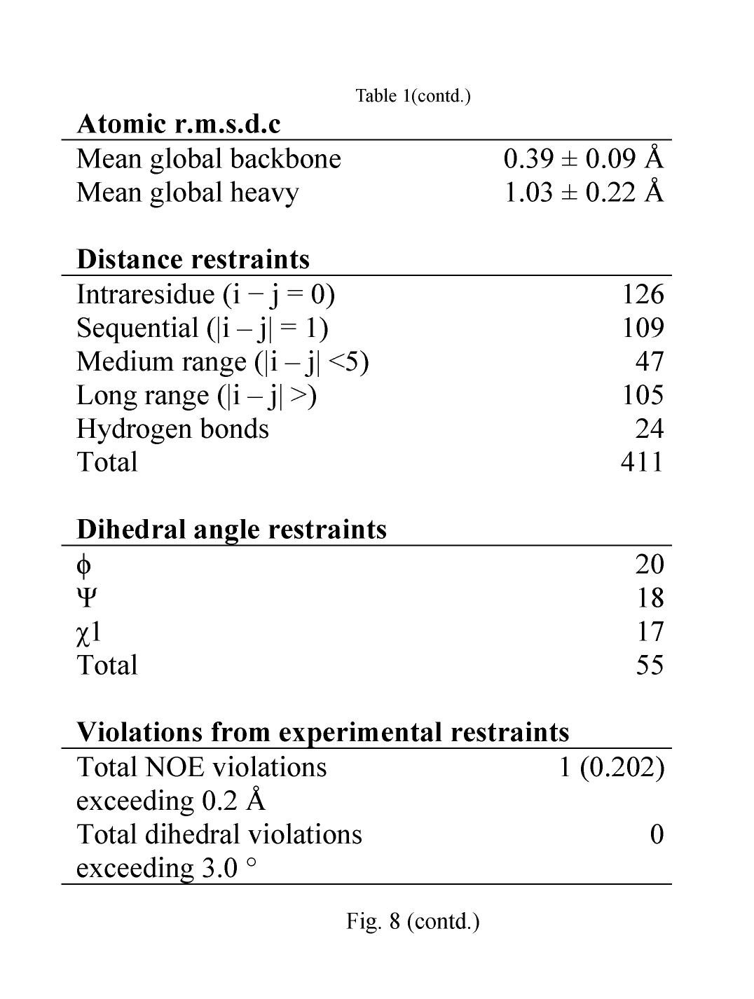

FIGS. 7A and B show the overlay of the 20 best structures of nemertide α-1 and a ribbon representation including disulfide bonds. The structure adapts a compact fold around the three disulfides located at the core of the molecule. Disulfides are interlocked in an inhibitory cystine knot (ICK) motif, with connections Cys2 to Cys16, Cys9-20, and Cys15-26. Sequence loops between cysteines are exposed at the surface of the molecule. A short α-helix in the loop between Cys II and III is the only example of secondary structure. The structure is well defined from the N-terminal with a more flexible C-terminal. A hydrophobic patch could be noticed including the aromatic residues F8, W22 and F24 in close proximity to each other. NMR statistics is summarized in Table 1 (FIG. 8).

Structural comparison of the solution NMR structure of α-1 against the Protein Data Bank through the DALI server (Holm L, Rosenstrim, P. Nucleic Acids Res. 2010, 38, W545-9) was performed. The closest match with respect to Z score (Z: 3.7, (rmsd: 1.1, 21% sequence identity) is the human liver-expressed antimicrobial peptide 2 (LEAP-2), followed by the spider toxins ω-HXTX-Hv1a Z3.4 (rmsd: 0.8, 30% sequence identity) and κ-TRTX-Scg1a Z: 3.3 (rmsd: 1.6, 20% sequence identity).

### Example 5: Relative Toxicity of Multiple Alpha-Nemertide Peptides to Crustaceans

Alpha-Nemertides were prepared as described in Example 2, above, or by manual synthesis 5 on 2-chlorotrityl resin as described in the Material and methods section below. Having demonstrated the potent toxicity of Nemertide α-1 using shore crabs, further experimentation assessed the toxicity of Nemertide α-1 as well as five other α-Nemertide peptides in a brine shrimp (Artemia salina) bioassay. The brine shrimp assay is commonly used as toxicity assay, but is particularly suitable in the current work as a model system for crustaceans. In the first round of experiments Nemertides α-1, α-2, α-5, and α-6 were evaluated. As seen in FIG. 9, Nemertide α-5 was even more potent than α-1. Nemertides α-2 and α-6 were slightly less potent but still highly toxic to crustaceans.

The IC50 for Nemertides α-1 and α-5 is less than 1 uM, whereas the IC50 for Nemertides α-2 and α-6 is one order of magnitude higher.

In the second round of experiments, Nemertides α-1, α-3, and α-4 were evaluated. As seen in FIG. 10, the activity of Nemertide α-4 was equivalent to α-1. Nemertide α-3 showed lower potency but still demonstrates high toxicity.

### Example 6: Structure-Activity Elucidation of Alpha-Nemertide Peptides

The sequences of the native peptides tested in the brine shrimp assays (see Example 5, above) differ at positions 4, 8, and 25 (see FIG. 3E). The most active peptides in the brine shrimp assay were Nemertides α-1, α-4, and α-5. Each of these has Phe at position 8 and a small residue (Ala or Ser) at position 4. For the peptides showing lower activity, Nemertide α-2 has a small Ala residue at position 4 but lacks Phe at position 8; Nemertide α-3 has a large, positively charged Lys at position 4 and also lacks Phe at position 8; and Nemertide α-6 also has a large, positively charged Lys in position 4 but does have Phe at position 8.

Common for all peptides evaluated is a Lys or His at position 25, indicating this portion may be of lesser importance, at least for crustacean toxicity.

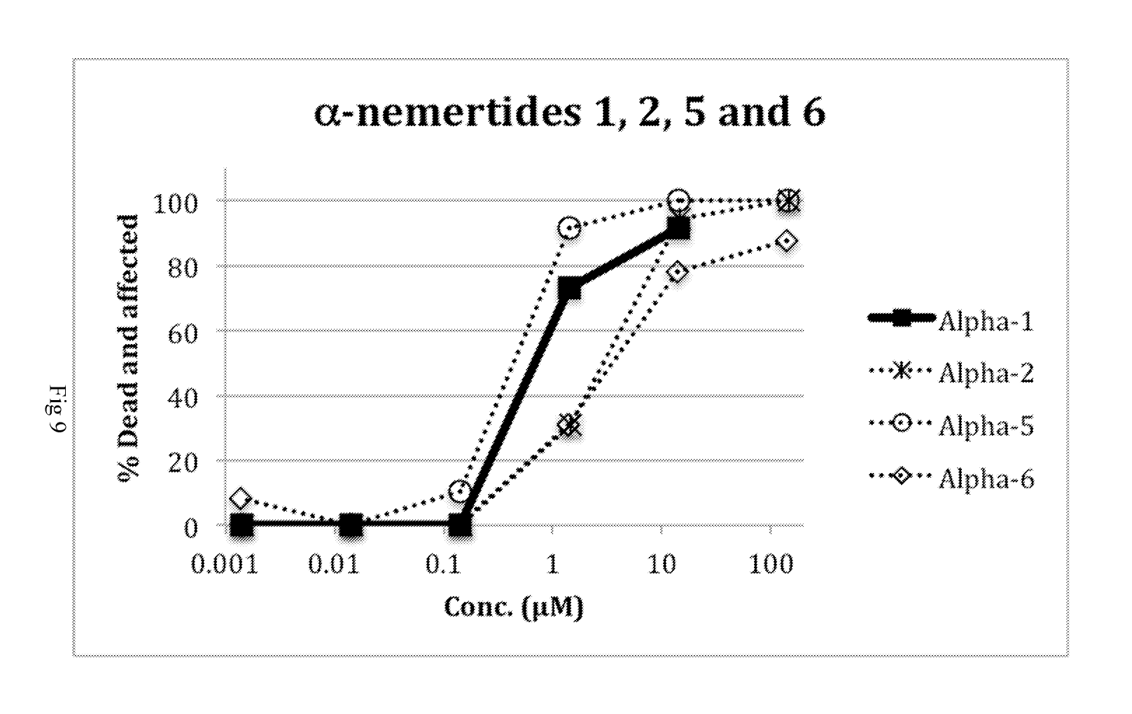

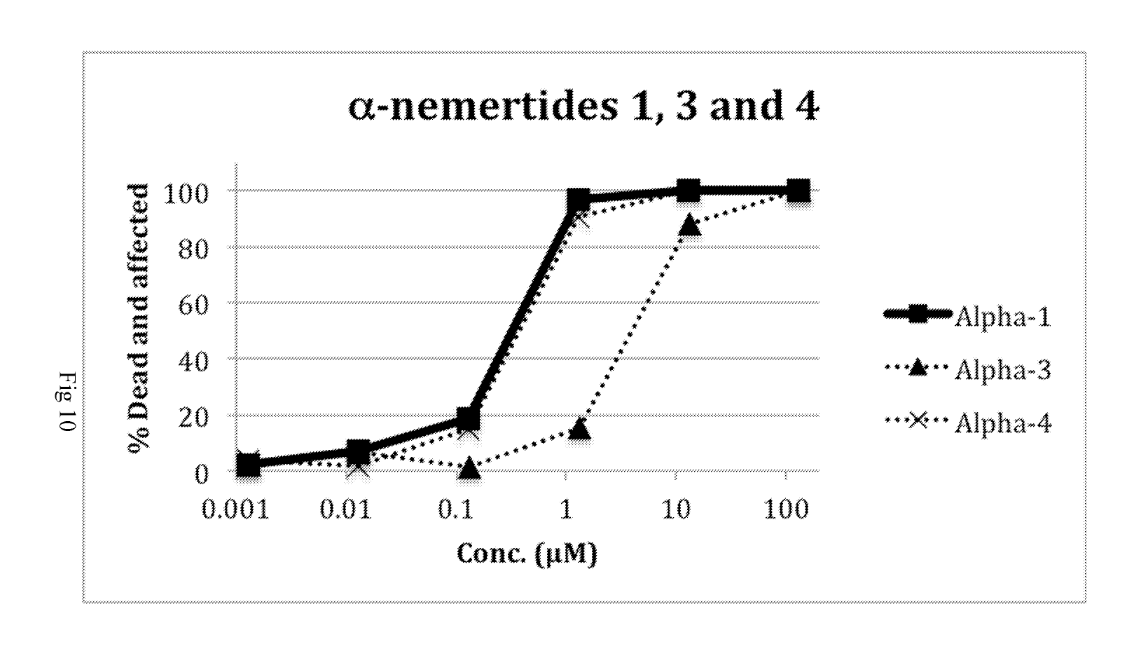

To further probe the structure-activity relationship, Nemertide α-1 was mutated at these positions with Ala. For that purpose, two full length peptides were prepared using solid phase peptides synthesis with Ala replacing the hydrophobic amino acids Ile at position 3 and Phe at position 8., As seen in FIG. 11, having N-terminal hydrophobic residues appears to be directly related to toxicity. Mutants I3A mutant and F8A that are lacking this feature had more than 100-fold lower activity than native Nemertide α-1, and they are essentially inactive in the brine shrimp assay.

In addition, mutants T5A and S7A were prepared both of which had a small residue (Thr, Ser, respectively) replaced by Ala. These mutants exhibited approximately 10-fold lower activity than Nemertide α-1 but they were active.

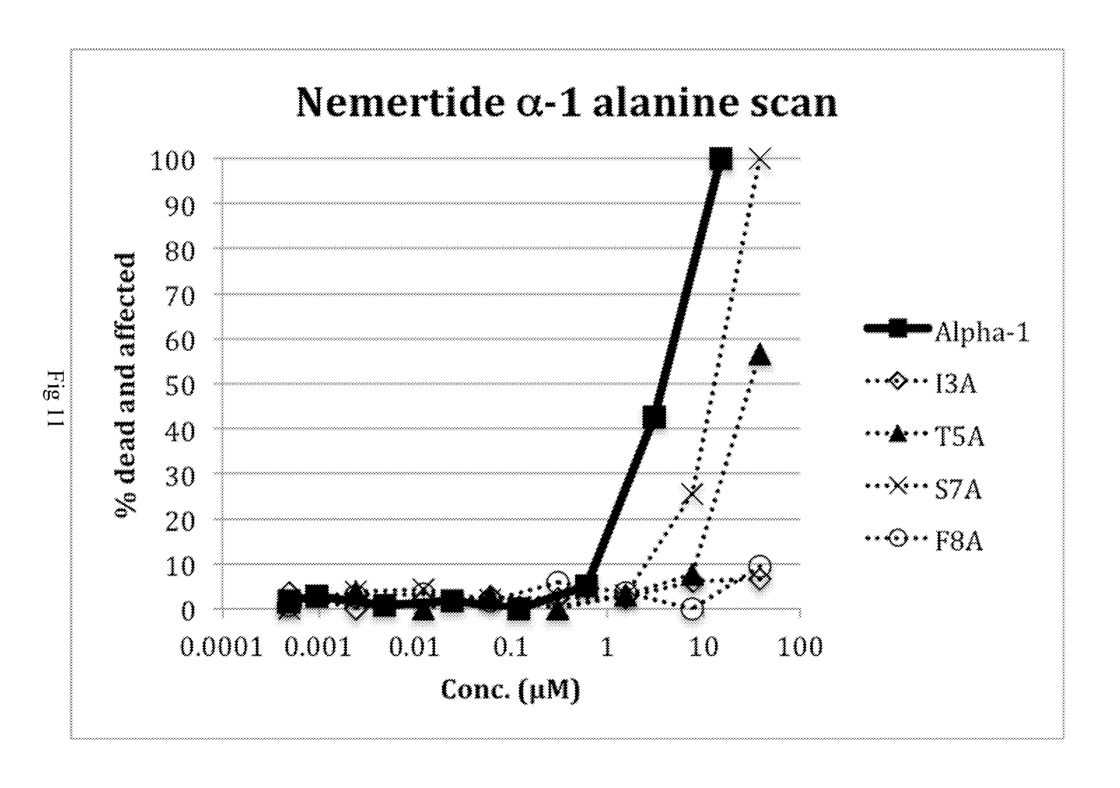

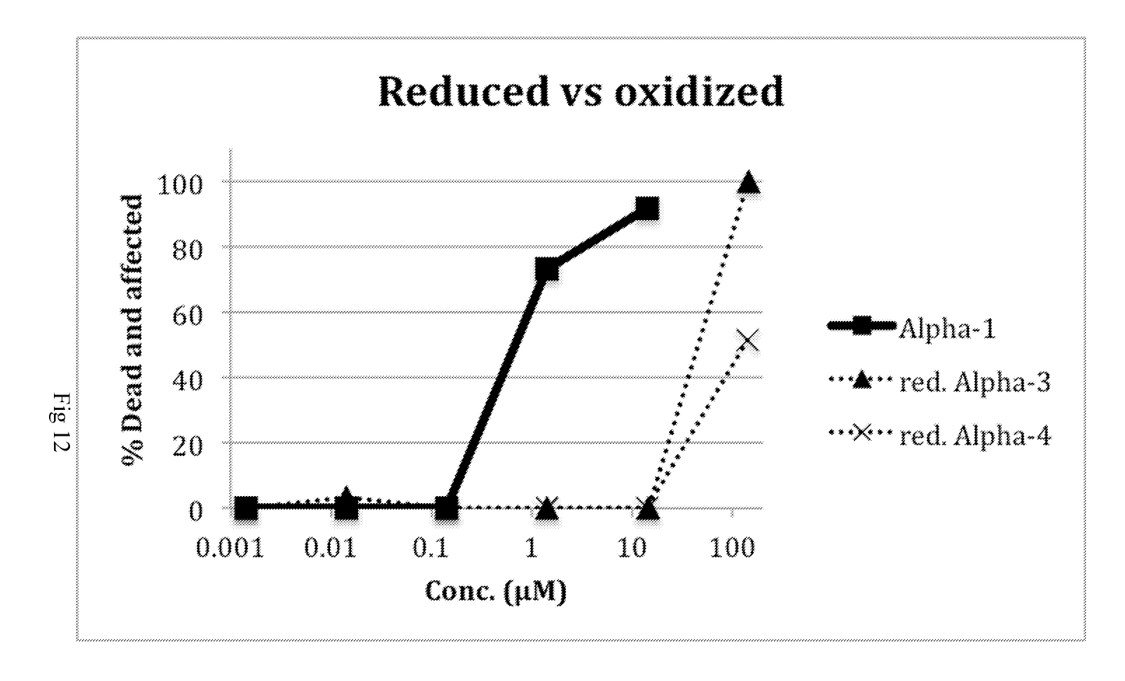

Having demonstrated Alpha-Nemertide positions and residues important for toxicity, a further experiment compared folded and unfolded peptides. Nemertides α-3 and α-4 were reduced using dithiotreitol to produce unfolded peptides. The experimental versions would comprise mixtures of misfolded, partially folded and possibly small amounts of correctly folded peptides. Their activity was evaluated against native folded Nemertide α-1 in the brine shrimp assay. As seen in FIG. 12, lack of native folding resulted in 10- to 100-fold lower activity.

Materials and Methods

Collection of Lineus longissimus. Sweden is the country of origin of the biological materials incorporated in the claimed invention. Living specimens of L. longissimus were collected in Swedish territorial waters on the west coast of Sweden (Kosterfjord, near the Sven Loven Center for Marine Sciences, Tjärnö, Sweden, 35 m depth) and identified by Dr Malin Strand, Tjärnö Loven Center. Mucus was collected by placing specimens in a small container containing seawater and gently agitating the animal with a glass rod. Mucus was then collected, and lyophilized. One specimen was cut into pieces, which were either flash frozen in liquid nitrogen or placed in RNA-later solution. The flash frozen samples were stored at −80° C. and the RNA-later preserved samples were stored at −20° C., after overnight storage at 4° C., until further processing.

Peptide Isolation. The lyophilized mucus from one collection was dissolved in 12.5 ml 30% acetonitrile (AcN) in water and 0.1% formic acid (FA). Aliquots of 2.5 ml were desalted using size exclusion chromatography (SEC; PD-10, GE Healthcare). The high molecular weight eluate was collected and lyophilized, before being redissolved in 10% AcN, 0.1% FA in water and subjected to RP-HPLC on a Phenomenex Jupiter column (5μ C18 300 Å, 250×4.6 mm) using a Shimadzu LC20 system equipped with a UV-detector. The gradient ranged from 5% AcN, 0.05% trifluoroacetic acid (TFA) to 55% AcN over 25 minutes. The three main peptides were subjected to quantitative amino acid analysis at the Amino Acid Analysis Center, Department of Biochemistry, Uppsala University.

Peptides were reduced and alkylated using dithiothreitol (DTT) and iodoacetamide (IAM), as reported previously. Alkylated peptides were desalted using SEC, and digested with trypsin, chymotrypsin and endoproteinase Glu-C, in separate experiments, prior to MS-sequencing. In short, dry, reduced and alkylated peptide was dissolved in 50 mM NH₄HCO₃ solution containing 4 μg/ml enzyme. The solution was incubated at 37° C. over night prior to LC-MS and LS-MSMS analyses.

LC-MS/MSMS. Peptides were analyzed using UPLC-QToF nanospray MS (Waters nanoAcquity, QToF Micro; 75 μm×250 mm 1.7 μm BEH130 C18). The LC gradient ranged from 1% to 90% AcN (0.1% FA) over 50 minutes at a flow rate of 0.300 μl/min. Detection was done in positive ion mode, and data was collected between m/z 200-2000. The mass spectrometer was operated under MassLynx v. 4.1. Data directed analysis (DDA) was used for MSMS. The survey scan window was set to 200-2500 m/z and MSMS scan to 50-2000 m/z. The collision energy profiles ranged from 25-70 V.

Total RNA Extraction and Generation of Transcriptomic Data. Total RNA was extracted from both flash frozen and samples stored in RNAlater®, using Qiagen AIIPrep DNA/RNA Mini Kit. The combined total RNA was sent to Macrogen (Korea) for Illumina HiSeq 2000 based RNA-seq paired end analysis, and assembled by Macrogen using Trinity (v 2011-11-26).

The assembled transcriptome was either translated into protein sequences using the EMBOSS getorf tool as utilized in the graphic user interface eBioX (v. 1.5.1), or for preparation of local nucleotide NCBI BLAST+ databases through Unipro uGENE's (v. 1.14.0) implementation of NCBI BLAST+. The sequenced tryptic/chymotryptic peptides were used as query in tBLASTn or BLASTp BLAST+ searches in the local L. longissimus transcriptome databases to confirm and complete the sequence.

The sequences, now annotated α-1, α-2 and β-1 were blasted against public generalistic databases (NCBI, UniProt) and the specialized databases Conoserver and Arachnoserver. The latter two databases are focused on toxin-like peptides from Conus spp. and arachnoids respectively. The ConoPrec tool from conoserver was used to predict and classify the full precursor sequences according to Conoserver standards.

Public transcriptomic data from Nemertea spp. data were downloaded from ftp://popphyl.univ-montp2.fr/contigs/Lineus. lacteus, L. longissimus, L. pseudolacteus, L. ruber, and L. sanguineus) (Romiguer J et al. Nature 2014, 515, 261-3); http://figshare.com/articles/Nemertean_Trinotate_annotation_reports/1203580 (accessed 2015-10-14) Cephalothrix hongkongiensis, Cephalothrix linearis, Cerebratulus marginatus, Lineus lacteus, Lineus longissimus, Lineus ruber, Malacobdella grossa, Paranemertes peregrina, and Tabulanus polymorphus) (Whelan N V et al. Genome Biol. Evol. 2015, 6, 3314-25). The assembled transcriptomes of Tabulanus punctatus, Carinoma hamanako, C. hongkongiensis, Hubrechtella. ijimai, Baseodiscus unicolor, C. marginatus, Riseriellus occultus, Argonemertes australiensis, M. grossa, Nipponemertes. sp., Paranemertes peregrine, and P. beebei were kindly provided by Andrade (Andrade S C S et al. Mol. Biol. Evol. 2014, 31, 3206-15). All sequences were combined in a single fasta file and the combined database was mined using BLAST+ and fuzz-pro/tran.

MALDI-Imaging. The frozen L. longissimus tissues were cut using a cryostat-microtome (Leica CM3050S; Leica Microsystems, Welzlar, Germany) at a thickness of 14 μm, thaw-mounted onto conductive indium tin oxide (ITO) glass slides (Bruker Daltonics), and stored at −80° C. Sections were dried gently under a flow of nitrogen and desiccated at room temperature for 15 min, after which they were imaged optically using a photo scanner (Epson perfection V500). The samples were then coated with 2,5-Dihydroxybenzoic acid (DHB) (35 mg/ml in 50% AcN, 0.2% TFA) using an automatic sprayer (TM-Sprayer; HTX Technologies, Carrboro, N.C.). MALDI-MSI experiments were performed using a MALDI-TOF/TOF (Ultraflextreme, Bruker Daltonics, Bremen Germany) mass spectrometer with a Smartbeam II 2 kHz laser in positive ion mode. The laser power was optimized at the start of each run and then held constant during the MALDI-MSI experiment. Purified peptides α-1, 2 and β-1 were spotted on one of the sections as an in-situ reference to establish the masses for the peptides in this system setup.

Peptide Synthesis. Nemertide α-1 was synthesised on a TentaGel XV HMPA resin (0.21 mmol/g) on 0.05 mmol scale using Fmoc based solid phase peptide synthesis (SPPS). Couplings of the first four C-terminal amino acids were carried out manually. The remaining residues was assembled using microwave assisted SPPS on a Liberty1 microwave peptide synthesizer (CEM Corp., Matthews, N.C.). Leu-Ser (residues 12 and 13) was introduced as a pseudoproline dipeptide to prevent peptide chain aggregation, and coupled manually. Due to the high swelling properties of the resin, 0.25 mmol scale of reagents (20 times excess), was used to ensure full coverage of the resin. The synthesis was repeated using 4 times excess of reagents but adjusting volume to cover the resin, to give 0.1 mmole of peptide on resin. After final Fmoc deprotection, peptide was cleaved and side chain protecting groups removed using a mixture of 95% TFA/2.5% triisopropylsilane (TIPS)/2.5% H₂O, (2 hrs, RT). TFA was removed under a stream of N₂, and peptide was precipitated with cold diethyl ether and collected by liquid-liquid extraction with 50% AcN/0.1% TFA. The aqueous layer was lyophilized.

Crude peptide was subjected to oxidative folding in a GSH:GSSG 2 mM: 0.4 mM in 0.1 M NH₄HCO₃ (pH 8.5), containing 20% isopropanol. The folding mixture was diluted to a final concentration of 6% isopropanol and the peptide or peptidomimetic was purified using RP-HPLC-UV using a Phenomenex Jupiter C18 column (250×10 mm 5μ) with a gradient from 5% AcN, 0.05% TFA to 97% in 45 minutes, at a flow rate of 4 ml/s. An aliquot of folded and purified peptide was co-injected with native α-1 into LC-MS to prove the conformity between the synthetic and native peptide.

Peptides α-2 to -6 and alanine mutants were synthesized in an analogous manner, using FMOC-based SPPS on HMPA or 2-chlorotrityl resins. Syntheses of some peptides (e.g. α-2 and the alanine mutants) were assembled manually, and without the use of the dipeptide. The folding protocol was optimized to include DMSO instead of isopropanol.

Chromatographic profiles of folding mixtures were similar to the ones for nemertide alpha-1, comprising a prominent sharp peak eluting as the last peak in the folding mixtures. The yields of this peak were approximately 50-60% as judged my HPLC-UV. These late eluting peaks were considered to have the correct ICK scaffold, and a native fold. No reference material was available for co-injection of native and synthetic peptides besides alpha-nemertide 1 and 2, but native folds were corroborated by the potent activity of peptides.

NMR Structure Determination. For NMR analysis synthesized α-1 was dissolved in 10% D₂O in H₂O, and data collected on a Bruker Avance 600 MHz spectrometer equipped with a cryoprobe. 2,2-Dimethyl-2-silapentane-5-sulfonate (DSS) was added and used as internal standard (0.0 ppm). Two-dimensional spectra (i.e. TOCSY, NOESY, ¹³CHSQC, ¹⁵NHSQC) were recorded at 298 K. TOCSY spectra were collected at five temperatures 288-308 with 5 K increments to establish temperature coefficients used for prediction of hydrogen bonds.

The NMR spectra were assigned in CARA as described before. In brief, CYANA 3.0 was used to automatically assign NOE couplings, generate distance restraints and calculate preliminary structures from the assigned and integrated peaks. CNS was used to refine the structures with regard to water molecules. MolProbity was used to evaluate the 50 structures with lowest overall energies, and the 20 structures with lowest scores and good covalent geometries were selected. Atomic RMSDC was calculated over the residues between the first and last cysteine residues using MOLMOL. Figures were prepared in PyMOL. The Dali server was used for comparing the NMR structure against structures in the Protein Data Bank (PDB) to related structures, regardless of sequence similarity. Default settings were used.

Carcinus maenas Assay. Shore crabs (20-50 g) were injected with control (500 μl sterile filtered sea water) or nemertide α-1 dissolved in sterile filtered sea water, into the cephalothorax between the first and second walking leg on the right side of the crab. Doses ranged from 0.1-50 μg/kg, in a maximal volume of 500 μl. The crabs were placed into a container filled with seawater and observed. All injections were made in duplicate.

Heterologous Expression in Xenopus Oocytes. Complementary DNA encoding the Naᵥ-channels was subcloned into the corresponding vector: the α-subunits rNaᵥ1.1/pLCT1(NotI), rNaᵥ1.4/pUI-2(NotI), hNaᵥ1.5/pcDNA3.1(XbaI), mNaᵥ1.6/pLCT(NotI), cockroach Blatella germanica BgNaᵥ1.1/pGH19(NotI), Drosophila melanogaster D m Naᵥ1, Verroa destructor VdNaᵥ1, and the corresponding β-subunits rβ1/pSP64T(EcoRI) and Drosophila melanogaster TipE/pGH19(NotI). The linearized plasmids—respective restriction enzymes are indicated in parentheses—were transcribed using the T7 (for rNaᵥ1.1, rNaᵥ1.4, mNaᵥ1.6, BgNaᵥ1.1, TipE) or the SP6 (for hNaᵥ1.5 and rβ1) mMESSAGE-mMACHINE transcription kit (Ambion, Austin, Tex.). The harvesting of stage V-VI oocytes from anaesthetized female Xenopus laevis frogs was previously described (28). Oocytes were injected with 50-70 nl of cRNA at a concentration of 1-3 ng/nl using a micro-injector (Drummond Scientific, Broomall, Pa.). The oocytes were incubated in an ND96 solution containing: NaCl, 96 mM; KCl, 2 mM; CaCl₂, 1.8 mM; MgCl₂, 2 mM and HEPES, 5 mM (pH 7.4), supplemented with 50 mg/l gentamycin sulfate and 0.5 mM theophylline. Oocytes were stored for 1-5 days at 16° C. until sufficient expression of Naᵥs was achieved.

Electrophysiology. Whole-cell currents from oocytes were recorded at room temperature (18-22° C.) by the two-electrode voltage clamp technique using a GeneClamp 500 amplifier (Molecular Devices, Sunnyvale, Calif.) controlled by a pClamp data acquisition system (Molecular Devices). Oocytes were placed in a bath containing ND96 solution. Voltage and current electrodes were filled with 3M KCl, and the resistances of both electrodes were kept between 0.7 and 1.5 MO). The elicited currents were sampled at 20 kHz and filtered at 2 kHz using a four-pole, low pass Bessel filter. To eliminate the effect of the voltage drop across the bath grounding electrode, the bath potential was actively controlled by a two-electrode bath clamp. Leak subtraction was performed using a −P/4 protocol. Whole-cell current traces were evoked every 5 s by a 100 ms depolarization to the voltage corresponding to the maximal activation of the Naᵥ-subtype in control conditions, starting from a holding potential of −90 mV. Concentration-response curves were constructed by adding different toxin concentrations directly to the bath solution. The percentage of Naᵥ blockade was plotted against the logarithm of the applied concentrations and fitted with the Hill equation: y=100/[1+(IC₅₀/[toxin])h], where y is the amplitude of the toxin-induced effect, IC₅₀ is the toxin concentration at half maximal efficacy, [toxin] is the toxin concentration and h is the Hill coefficient. To investigate the effects on the voltage dependence of activation, current traces were induced by 100-ms depolarizations from a holding potential of −90 to 65 mV with 5-mV increments. To investigate the effects on the steady-state inactivation process, oocytes were depolarized using a standard two-step protocol. From a holding potential of −90 mV, 100-ms prepulses were generated, ranging from −90 to 65 mV with 5-mV increments, immediately followed by a 100-ms test pulse to −10 mV. Data were normalized to the maximal Na⁺ current amplitude, plotted against prepulse potential and fitted using the Boltzmann equation: I_{Na}/Iₘₐₓ=[(1−C)/(1+exp((V−Vₕ)/k))]+C, where Iₘₐₓ is the maximal I_{Na}, Vₕ is the voltage corresponding to half-maximal inactivation, V is the test voltage, k is the slope factor, and C is a constant representing a non-inactivating persistent fraction (close to zero in control). Comparison of two sample means was made using a paired Student's t test (p<0.05). All data was analyzed using pClamp Clampfit 10.0 (Molecular Devices®, Downingtown, Pa.) and Origin 7.5 software (Originlab®, Northampton, Mass.) and is presented as mean±standard error (S.E.M) of at least 3 independent experiments (n≥3).

Artemia salina Assay of Alpha-Nemertides. 100 μl of sea water containing about 20-30 brine shrimps were added to each experimental well of a 96-well microplate. 100 μl of peptide solutions were then added to each well. As a control, some wells were filled with 100 μl of seawater containing shrimps and 100 μl milliQwater. The plates were covered and incubated in the dark at room temperature for 24 hours. After 24 hours, 10 second video clips of each well were filmed through a microscope. The shrimps were subsequently counted and categorized as either dead (not moving), affected (moving slowly, mostly at the bottom of the well) or normal (moving normally compared to the control at all levels of the well). The shrimps were then immobilized by addition of 100 μl of methanol. After 1 hour the total number of shrimps in each well was counted and the ratio (dead+affected)/total was calculated.

1-16. (canceled)

17. An isolated peptide or peptidomimetic, comprising an α-nemertide moiety, wherein:
a) said α-nemertide moiety has a sequence according to SEQ ID NO:1; or
b) said α-nemertide moiety has a sequence differing from SEQ ID NO: 1 by residue substitutions, deletions or insertions numbering no more than 9 in total.

18. The peptide or peptidomimetic according to claim 17, wherein said α-nemertide moiety has a sequence differing from SEQ ID NO: 1 by residue substitutions, deletions or insertions numbering no more than 6 in total.

19. The peptide or peptidomimetic according to claim 17, wherein the α-nemertide moiety comprises six C residues at positions aligning with the positions of C residues in SEQ ID NO: 1.

20. The peptide or peptidomimetic according to claim 17, wherein said α-nemertide moiety has a sequence according to the consensus according to SEQ ID NO: 2.

21. The peptide or peptidomimetic according to claim 17, wherein the α-nemertide moiety comprises a disulphide bridge between C residues located at positions aligning with the positions 2 and 16 of SEQ ID NO: 1, a disulphide bridge between C residues located at positions aligning with the positions 9 and 20 of SEQ ID NO: 1, and/or a disulphide bridge between C residues located at positions aligning with the positions 15 and 26 of SEQ ID NO: 1.

22. The peptide or peptidomimetic according to claim 17, wherein the α-nemertide moiety comprises a disulphide bridge between C residues located at positions aligning with the positions 2 and 16 of SEQ ID NO: 1, a disulphide bridge between C residues located at positions aligning with the positions 9 and 20 of SEQ ID NO: 1, and a disulphide bridge between C residues located at positions aligning with the positions 15 and 26 of SEQ ID NO: 1.

23. The peptide or peptidomimetic according to claim 17, wherein the peptide or peptidomimetic is a peptide having a sequence comprising at least one difference compared to any naturally occurring peptide sequence.

24. A method of treatment of a disease, comprising administering an effective amount of a peptide or peptidomimetic according to claim 17 to a subject in need thereof.

25. The method according to claim 24, wherein said α-nemertide moiety has a sequence differing from SEQ ID NO: 1 by residue substitutions, deletions or insertions numbering no more than 6 in total.

26. The method according to claim 24, wherein the α-nemertide moiety comprises six C residues at positions aligning with the positions of C residues in SEQ ID NO: 1.

27. The method according to claim 24, wherein said α-nemertide moiety has a sequence according to the consensus according to SEQ ID NO: 2.

28. The method according to claim 24, wherein the α-nemertide moiety comprises a disulphide bridge between C residues located at positions aligning with the positions 2 and 16 of SEQ ID NO: 1, a disulphide bridge between C residues located at positions aligning with the positions 9 and 20 of SEQ ID NO: 1, and/or a disulphide bridge between C residues located at positions aligning with the positions 15 and 26 of SEQ ID NO: 1.

29. The method according to claim 24, wherein the α-nemertide moiety comprises a disulphide bridge between C residues located at positions aligning with the positions 2 and 16 of SEQ ID NO: 1, a disulphide bridge between C residues located at positions aligning with the positions 9 and 20 of SEQ ID NO: 1, and a disulphide bridge between C residues located at positions aligning with the positions 15 and 26 of SEQ ID NO: 1.

30. A method of pest control, comprising administering an effective amount of a peptide or peptidomimetic according to claim 17 to pests or their environment.

31. The method according to claim 30, wherein said α-nemertide moiety has a sequence differing from SEQ ID NO: 1 by residue substitutions, deletions or insertions numbering no more than 6 in total.

32. The method according to claim 30, wherein the α-nemertide moiety comprises six C residues at positions aligning with the positions of C residues in SEQ ID NO: 1.

33. The method according to claim 30, wherein said α-nemertide moiety has a sequence according to the consensus according to SEQ ID NO: 2.

34. The method according to claim 30, wherein the α-nemertide moiety comprises a disulphide bridge between C residues located at positions aligning with the positions 2 and 16 of SEQ ID NO: 1, a disulphide bridge between C residues located at positions aligning with the positions 9 and 20 of SEQ ID NO: 1, and/or a disulphide bridge between C residues located at positions aligning with the positions 15 and 26 of SEQ ID NO: 1.

35. The method according to claim 30, wherein the α-nemertide moiety comprises a disulphide bridge between C residues located at positions aligning with the positions 2 and 16 of SEQ ID NO: 1, a disulphide bridge between C residues located at positions aligning with the positions 9 and 20 of SEQ ID NO: 1, and a disulphide bridge between C residues located at positions aligning with the positions 15 and 26 of SEQ ID NO: 1.
